(19) United States
(12) Patent Application Publication
(10) Pub. No.: US 2011/0087173 A1
(43) Pub. Date: Apr. 14, 2011

(54) AUTOMATIC SYRINGES

(76) Inventors: Wilmer L Sibbitt, JR., Albuquerque, NM (US); Randy R. Sibbitt, Helena, MT (US)

(21) Appl. No.: 12/899,543

(22) Filed: Oct. 7, 2010

Related U.S. Application Data
(60) Provisional application No. 61/250,671, filed on Oct. 12, 2009.

(51) Int. Cl. A61M 5/315; A61M 5/31; B23P 17/04

(52) U.S. Cl. 604/207; 604/227; 29/428

(57) ABSTRACT

The present invention provides automatic syringes that can be held with one hand in a pencil-grip in which the fingers (thumb or index finger) operate a forwardly based valve that along with selection of needle diameter and length, control the ingress or egress of fluid from the automatic syringe. These finger-controlled one-handed automatic syringes can be used in image-directed procedures such as ultrasound (sonography)-directed procedures in which the ultrasound probe is controlled with one hand and the automatic syringe with the other, although many other applications are readily apparent to those skilled in the art including biopsy, epidural injections, administration of anesthesia, and detection of blood vessels and body cavities as well as other applications.

### CROSS REFERENCE TO RELATED APPLICATIONS

This application claims priority to U.S. provisional 61/250,671 filed 12 Oct. 2009, which is incorporated herein by reference.

### TECHNICAL FIELD

The present invention relates to the field of syringes and more specifically to automatic syringes that can be operated with one hand.

### BACKGROUND

Image-guided procedures particularly utilizing ultrasound (sonography) are becoming a mainstay of medicine both within and outside of radiology, including the fields of obstetrics-gynecology (prenatal medicine, amniocentesis, chorionic villus sampling), intensive care (placement of central lines, thoracentesis, paracentesis, regional nerve blocks), breast medicine (biopsy and cyst aspiration), endocrinology (thyroid biopsy and cyst aspiration), anesthesiology (placement of central lines, regional nerve blocks, epidural injections), neurology (intramuscular injection of botulinum toxin), pain medicine (injection of nerve and facets, and nerve oblation procedures), and orthopedic surgery and rheumatology (injection and aspiration of joints and related structures). There are two major methods for using sonography for syringe and needle procedures: 1) where the operator controls the syringe and needle and an assistant controls the ultrasound probe, or 2) where the operator controls the syringe and needle AND controls the ultrasound probe. In many situations, there is not an assistant; thus, it is very advantageous to have a syringe that can be operated with one hand. Moreover, when using ultrasound with one operator controlling both the probe and syringe, it can be advantageous to hold the syringe like a pencil so that the hand rests on the patients skin, and the depth and penetration of the needle is controlled by holding the syringe and needle like a pencil, with the forward section of the syringe held between the thumb, index finger and middle finger. Of course, in this manual arrangement, the plunger of the syringe which is at the back section of syringe, cannot be operated to aspirate or inject without radically changing finger position, thus, the need for automatic syringes to aspirate or inject that are controlled by a mechanism at the forward section of the syringe.

Automatic syringes that automatically inject when triggered were developed to make-self-injection simpler for patients, and for those situations where a medication must be injected rapidly with one hand, as in emergencies and in animal science.

US2005/222539A1 to Gonzales, U.S. Pat. No. 6,086,562 to Jacobsen et al., U.S. Pat. No. 4,258,713 and U.S. Pat. No. 4,378,015 to Wardlaw, U.S. Pat. No. 5,167,632 to Eid et al., U.S. Pat. No. 6,099,503 to Stradella, U.S. Pat. No. 4,601,708 to Jordan, U.S. Pat. No. 4,723,937 to Sarnoff, U.S. Pat. No. 6,280,421 and U.S. Pat. No. 6,280,421B1 to Kirchhofer et al., U.S. Pat. No. 6,575,939B1 to Brunei et al., U.S. Pat. No. 3,964,481 to Gourlandt et al., U.S. Pat. No. 4,529,403, U.S. Pat. No. 4,573,971, U.S. Pat. No. 4,573,972 to Kamstra, U.S. Pat. No. 6,830,560 to Gross et al., US2007/167920A1, U.S. Pat. No. 4,723,937 to Sarnoff, US2007/167920A1 and US2006/270984A1 to Hommann, U.S. Pat. No. 6,149,626 to Bachynsky, US2006/178642A1 to Gillespie et al., demonstrate automatic spring, gas, motor, or solenoid driven injection devices without any finger-operated anterior control valve, and is not held in a finger-grip position. Most of these devices utilize and on-off trigger so that injection occurs once the trigger is disengaged and the injection proceeds through the entire cycle until the syringe is empty or reaches a prefixed “stop”.

US 20070142766, US 20070244446, US 20070232993, and US 20070213674 to Sundar et al. demonstrate a spring-driven injection device that is used to detect body cavities, and the epidural space in particular. This device does not have a valve, cannot automatically aspirate, cannot provide controlled injection, the plunger does not lock, and the syringe is unstable when filled with liquid (that is it automatically injects).

U.S. Pat. No. 4,261,358 to Vargas et al. describes an automatic injection syringe that provides a mechanical mechanism for coiling the spring so that the device can inject. The spring is shown along the plunger or at the end of the plunger and at the end of the plunger. There is no anterior valve or other means to control outflow or inflow into the device.

U.S. Pat. No. 4,364,376 to Bigham describes a syringe with a valve (turncock or stopcock) but it is a double bolus syringe for injection, requires two hands to operate, and is not an automatic syringe.

U.S. Pat. No. 4,711,250 to Gilbaugh et al. describes a handle that applies pressure to the plunger by ways of an external spring and thus can automatically inject. However, the syringe is not held in the fingertips, and there is no finger-operated valve. U.S. Pat. No. 4,755,172 and U.S. Pat. No. 4,863,429 to Baldwin describes an external housing that converts a syringe into automatic infusion device with an anteriorly biased on-off valve; however, this not a hand-held device, is bulky, is not intended for precise placement of medication, and cannot be used to aspirate.

U.S. Pat. No. 5,246,011 and U.S. Pat. No. 5,651,372 to Caillouette describe an aspiration syringe that has a ratchet mechanism or stop to control the plunger, but it has no valve connected to the needle, does not automatically inject, and cannot conveniently be held like a pencil.

U.S. Pat. No. 4,989,614 and U.S. Pat. No. 5,060,658 to Dejter et al. demonstrate a finger-operated aspiration device that is controlled by a finger operated valve or solenoid that opens and closes to a syringe that is held in an aspiration phase by a posteriorly biased spring system. In several embodiments a spring(s) drives the plunger mechanism but is external to the syringe itself. U.S. Pat. No. 4,549,554 and U.S. Pat. No. 4,693,257 to Markham reveal a finger operated aspiration device with vacuum provided by a rearwardly biased spring or by stops or pins on the plunger that lock the plunger in a vacuum position.

U.S. Pat. No. 3,833,000 to Bridgeman reveals an aspiration device with a proximal valve, finger operated, but no syringe and no ability to inject. These devices are “on” and “off”, and do not have any variable control as to vacuum, thus, they are not well-suited to fluid aspiration procedures where control of vacuum critical, and they are very expensive and complex, and they cannot be used to inject.

U.S. Pat. No. 4,236,516 to Nilson demonstrates a spring-driven automatic aspiration device, but there is no finger-controlled valve in at the proximal end of the syringe and this device cannot automatically inject.

U.S. Pat. No. 3,996,923 to Guerra, U.S. Pat. No. 4,326,541 to Eckels et al., U.S. Pat. No. 4,972,843 to Broden, and U.S. Pat. No. 4,509,534 to Tassin, U.S. Pat. No. 5,090,420 February 1992 Nielsen 128/764, and U.S. Pat. No. 4,643,198 to Ballies describe a blood drawing device with a forward valve to control vacuum, but uses vacuum tubes and not syringes, and the device is not an automatic injection syringe. U.S. Pat. No. 4,073,288 to Chapman also discloses a valve to control vacuum, but the valve is not situated at the front end of the syringe, and is a valve not easily manipulated by one finger.

US25101879A1 to Shidham et al. discloses an aspiration syringe with an anterior valve in the shape of a turn cock or stop cock that uses piston locks that snap onto the plunger to hold the plunger in place; however, this is not an injection automatic syringe, and the valve is “off-on” without variable adjustment ability.

U.S. Pat. No. 5,413,115 to Baldwin and U.S. Pat. No. 5,655,541 to Vattuone describes a biopsy syringe with a slide valve and a plunger latch or spacer to keep vacuum, however, this is an on-off valve without variation, and is not an automatic injection syringe. This valve is operated with the index finger, but is one-way valve and vents to the atmosphere.

U.S. Pat. No. 5,830,152 to Toa describes a syringe hold with a spring than converts a conventional syringe to an aspiration device. However, there is not an anterior valve, and the syringe cannot automatically inject.

U.S. Pat. No. 4,098,276 to Bloom et al. describes a syringe with a posteriorly biased spring on the plunger for aspiration and a double check valve, however, this valve is not finger-operated and this syringe functions as a reciprocating hand-operated pump, not an automatic aspiration or injection device. Moreover flow of fluid through the needle and syringe reciprocates or alternates in direction consistent with its pump function U.S. Pat. No. 4,427,015 to Redeauz describes an aspiration syringe with a valve, but the valve is distal not proximal on the syringe, there is no plunger, and vacuum is not used. U.S. Pat. No. 6,972,008 and U.S. Pat. No. 7,198,619 to Bills et al. describe an injection syringe with an anterior valve, but it is not an automatic syringe, and the valve is not easily operated with one finger.

U.S. Pat. No. 7,226,435 to Darnel and US2006/047250A1 to Hickingbotham et al. disclose an automatic injection syringe with a forward valve and a spring driven plunger; however, these devices use vials or ampoules and do not have conventional luer-based fittings, and cannot aspirate.

U.S. Pat. No. 5,176,642 to Clement describes a spring driven syringe that can both aspirate and inject, but it requires pressure and vacuum ports to actuate the device, and the trigger device is on the rear of the syringe on the plunger thus, the device is not controlled at the front of the syringe. U.S. Pat. No. 4,073,288 to Chapman is an aspiration syringe that is controlled with a valve, but cannot inject. U.S. Pat. No. 4,236,516 to Nilsson is an aspiration syringe, but does not have a valve.

U.S. Pat. No. 4,098,276 is a syringe with a forward valve and a spring, but this syringe is a refilling syringe used for pumping.

There are a number of aspiration syringes that utilize a forward placed valve system. U.S. Pat. No. 3,833,000 and U.S. Pat. No. 3,939,835 to Bridgman and U.S. Pat. No. 3,996,923 to Guerra reveal a bulb-style aspiration device with a forward valve, however, this type of valve can be difficult to operate with one finger, the device does not have a plunger system, and the device cannot inject.

An injection syringe comprising a spring surrounding the plunger and within the barrel has been proposed, including U.S. Pat. No. 3,964,481 to Gourlandt, U.S. Pat. No. 4,258,713 to Wardlaw, U.S. Pat. No. 461,358 to Vargas, U.S. Pat. No. 459,403 to Kamstra, U.S. Pat. No. 4,723,937 to Sarnoff, U.S. Pat. No. 5,167,632 to Eid, U.S. Pat. No. 5,176,642 to Clement, and U.S. Pat. No. 6,899,699 B2 to Enggaard. Some references disclose an injection syringe comprising a spring external to the plunger and compelling the syringe to inject including U.S. Pat. No. 4,755,172 to Baldwin and U.S. Pat. No. 6,099,503 to Stradella. Some references disclose an aspiration syringe where the plunger is fixed in position by a plunger lock, ratchet, latch, or other mechanism including U.S. Pat. No. 4,549,553 to Markham, U.S. Pat. Nos. 5,246,011 and 5,651,372 to Caillouette, U.S. Pat. No. 5,413,115 to Baldwin, U.S. Pat. No. 5,655,541 to Vattuone, U.S. Pat. No. 5,830,152 to Liang-Che Toa, and US2005/101879A1 to Shidham. However, the prior art does not describe an automatic syringe that is held in the pencil grip, can both automatically aspirate and inject using mechanical mechanisms, is controlled by a finger-operated valve at the front of the syringe, and can use conventional luer fittings and attachments. U.S. Pat. No. 4,819,684 to Zauge et al., U.S. Pat. No. 5,203,769 to Clement et al., U.S. Pat. No. 5,285,805 to Proper, U.S. Pat. No. 5,865,812 to Correia, U.S. Pat. No. 6,972,008 and U.S. Pat. No. 7,198,619 to Bills e al., U.S. Pat. No. 7,226,435 to Darnel, U.S. Pat. No. 6,569,117 to Ziv et al. and U.S. Pat. No. 5,046,528 to Manska disclose a type of valve that could be operated with one finger on such an aspiration injection syringe device.

Image-guided procedures particularly utilizing ultrasound (sonography) are becoming a mainstay of medicine both within and outside of radiology, including the fields of obstetrics-gynecology (prenatal medicine, amniocentesis, chorionic villus sampling), intensive care (placement of central lines, thoracentesis, paracentesis, regional nerve blocks), breast medicine (biopsy and cyst aspiration), endocrinology (thyroid biopsy and cyst aspiration), anesthesiology (placement of central lines, regional nerve blocks, epidural injections), neurology (intramuscular injection of botulinum toxin), pain medicine (injection of nerve and facets, and nerve oblation procedures), and orthopaedic surgery and rheumatology (injection and aspiration of joints and related structures). There are two major methods for using sonography for syringe and needle procedures: 1) where the operator controls the syringe and needle and an assistant controls the ultrasound probe, or 2) where the operator controls the syringe and needle AND controls the ultrasound probe. In many situations, there is not an assistant; thus, it is very advantageous to have a syringe that can be operated with one hand. Moreover, when using ultrasound with one operator controlling both the probe and syringe, it can be advantageous to hold the syringe like a pencil so that the hand rests on the patients skin, and the depth and penetration of the needle is controlled by holding the syringe and needle like a pencil, with the forward section of the syringe held between the thumb, index finger and middle finger. Of course, in this manual arrangement, the plunger of the syringe which is at the back section of syringe, cannot be operated to aspirate or inject without radically changing finger position, thus, the need for automatic syringes to aspirate or inject that are controlled by a mechanism at the forward section of the syringe.

### SUMMARY OF THE INVENTION

The present invention provides a one-hand operated automatic syringe that can both automatically aspirate and inject where both aspiration and injection is controlled using a forwardly mounted finger-operated valve and restraint of the plunger using a plunger locking mechanism. Such an automatic device is suitable for multiple purposes, and can be used for controlled suction biopsy, fine needle aspiration biopsy, aspiration of body fluids into the syringe, aspiration of medications into the syringe, and automatic injection of fluid and medications into the body or automatic expulsion of biopsy specimens out of a needle. Other uses include image-directed procedures, including ultrasound, administration of regional nerve blocks, and detection of body compartments, such as epidural injections.

The prior art does not describe an automatic syringe that can conveniently be held in a pencil grip, can both automatically aspirate and inject using mechanical mechanisms, is controlled by a finger-operated valve at the front of the syringe, and can use conventional luer fittings and attachments.

An example embodiment of this invention provides a multipurpose one-handed pencil-grip automatic syringe device that can both automatically aspirate and inject comprising a forwardly mounted finger controlled valve that operates the device, a largely conventional syringe barrel, a plunger modified to accommodate a spring within the syringe barrel, a means to retain the spring within the barrel, a spring biased to force the plunger into the syringe barrel to cause injection, a means to reversibly and mechanically engage the barrel to the plunger in order to lock the plunger to create vacuum and in order release the plunger so that the syringe only aspirates or injects when intended, and a device that can accommodate standard luer or luer lock fitting or with modification other fittings.

An example embodiment of this invention provides a multipurpose one-handed pencil-grip automatic syringe device that can automatically inject and can manually aspirate comprising a forwardly mounted finger controlled valve that operates the device, a largely conventional syringe barrel, a plunger modified to accommodate a spring within the syringe barrel, a means to retain the spring within the barrel, and a device that can accommodate standard luer or luer lock fitting or with modification other fittings.

An example embodiment of this invention provides a multipurpose one-handed pencil-grip automatic syringe device that can automatically aspirate and can manually inject comprising a forwardly mounted finger controlled valve that operates the device, a largely conventional syringe barrel, a means to reversibly and mechanically engage the barrel to the plunger in order to lock the plunger to create vacuum and in order release the plunger so that the syringe only aspirates or injects when intended, and a device that can accommodate standard luer or luer lock fitting or with modification other fittings. An example embodiment has a notched asymmetrical plunger that can rotate into a corresponding mating part on the barrel and another version that uses a spacer that holds the plunger in position.

Devices according to the present invention can be used for many applications including aspiration biopsy, administration of anesthesia, aspiration of body fluids, hydrodissection, location of body cavities, epidural location and injection, and many other medical procedures.

### BRIEF DESCRIPTION OF FIGURES

FIG. 1 is a schematic illustration of an example embodiment of an automatic syringe.

FIG. 2 is a schematic illustration of an assembled automatic syringe.

FIG. 3 is a schematic illustration of an assembled automatic syringe ready for use with the valve closed, the spring compressed, and the plunger locked, creating vacuum in the syringe barrel.

FIG. 4 is a schematic illustration of an automatic syringe with valve closed, the spring compressed and urging the stopper and plunger forward, the barrel filled with liquid, and the plunger unlocked creating pressurized liquid in the syringe barrel.

FIG. 5 is a schematic illustration of an automatic syringe with the valve open, the spring uncoiled and urging the stopper forward, the plunger unlocked, and the barrel empty of liquid, it having been expelled out the front of the syringe.

FIG. 6 is a schematic, close-up, illustration of an automatic syringe plunger illustrating the reversible interaction of the plunger notches with the endplate.

FIG. 7 is a schematic illustration of an automatic syringe held in a one-handed fashion in the pencil grip with the thumb-operated valve.

FIG. 8 is a schematic illustration of an automatic syringe without a spring.

FIG. 9 is a schematic illustration of an example embodiment of an aspiration syringe with a plunger spacer lock.

FIG. 10 is a schematic illustration of an example embodiment of an aspiration syringe with plunger spacer lock in place.

FIG. 11 is a schematic illustration of an example embodiment of a plunger lock.

FIG. 12 is a schematic illustration of an example embodiment of a plunger lock.

FIG. 13 is a schematic illustration of an example embodiment of a plunger lock.

FIG. 14 is a photograph of an example embodiment similar to that described in connection with FIG. 1.

FIG. 15 is a photograph of an example embodiment similar to that described in connection with FIG. 2.

FIG. 16 is a series of photographs of an example embodiment similar to that described in connection with FIGS. 3-5.

FIG. 17 is a photograph of an example embodiment similar to that described in connection with FIG. 6.

FIG. 18 is a photograph of an example embodiment of the present invention in use.

### DESCRIPTION OF THE INVENTION

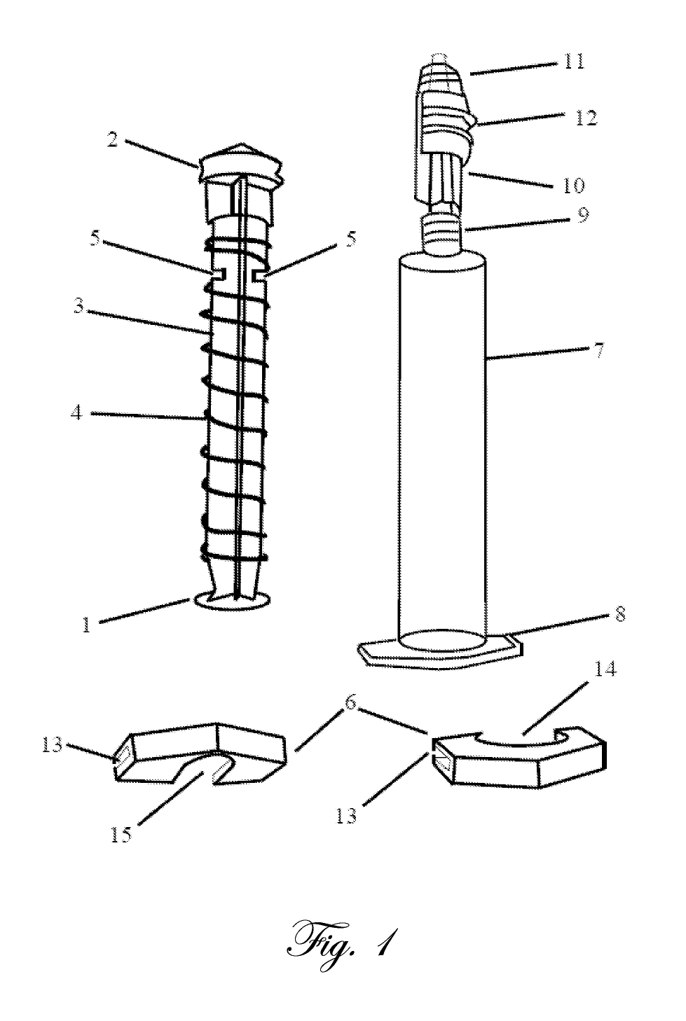

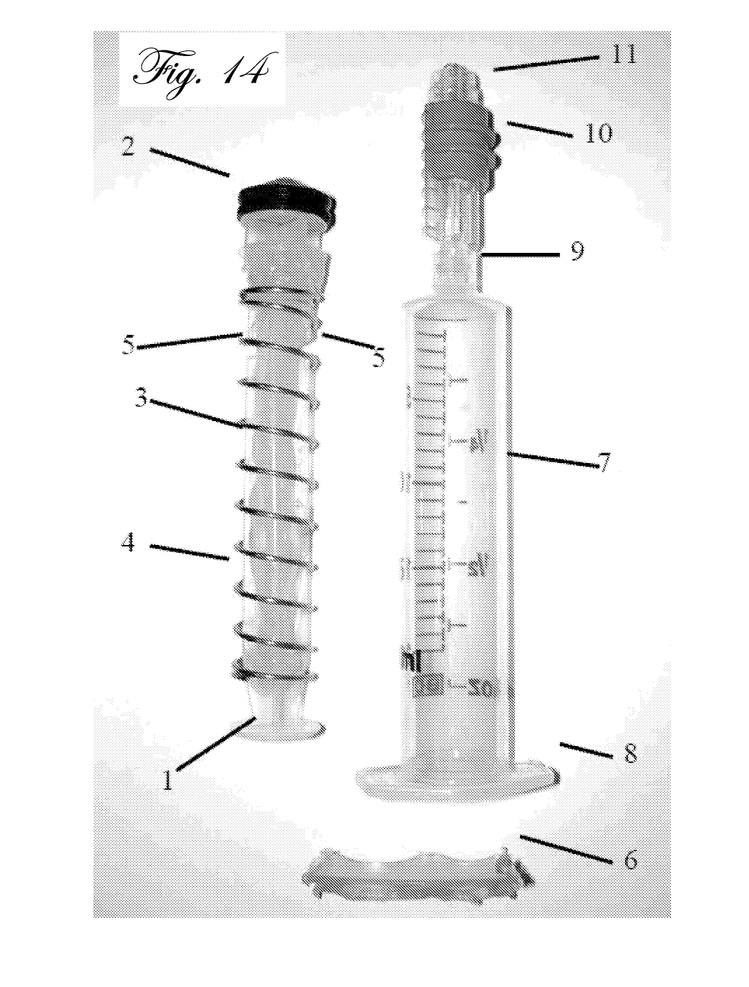

FIG. 1 is a schematic illustration of an example embodiment of an automatic syringe. FIG. 14 is a photograph of an example embodiment similar to that described in connection with FIG. 1. A plunger has a the thumb rest 1 (plunger flange), a the stopper or seal complex 2 of the plunger of a particular diameter, a plunger body 3 of a lesser diameter of the stopper or seal of the plunger, a spring 4 that encircles the plunger body and abuts on the seal complex that is of greater diameter than the spring, and notches 5 in the plunger that are the female component to the male component of a barrel endplate mechanism to reversibly lock the plunger in position. An endplate 6 of the barrel comprises a clip, fitting, or intrinsic molding of the barrel that is clipped, bonded, glued, welded or molded firmly to finger flanges 8 of the barrel 7 and contains and restrains the spring 4 within the syringe barrel 7 and reversibly mates with the notches 5 in the body of the plunger reversibly locking the plunger in position. The endplate provides two functions: 1) constrains the spring within the barrel, and 2) locks the plunger when desired.

In the example in the figure, the endplate 6 is a clip with a slit-like structure 13 to receive and bond the finger flanges, a structure to accommodate the barrel 14, and a more narrow structure 15 to accommodate and reversibly lock the plunger. The endplate can be constructed of one or many pieces to the same effect. For example in some prototypes a simple flat endplate was cemented on each finger flange to provide the same function as the clip 6 in the figure. The clip 6 can convert a conventional syringe barrel to an automatic syringe barrel, but dedicated automatic syringe barrels are also possible made of integrated or bonded parts. The endplate can permit the plunger to freely move in and out of the barrel in one position, and can then restrain the plunger when the plunger is rotated, most obviously at 45%, but lesser or greater angles can also be suitable depending on the mating structures on the plunger and endplate respectively. In one position the plunger 3 can move freely in and out of the barrel 7 and is not restrained by the endplate 6; however, when rotated about 45 degrees the notches 5 in the plunger reversibly mate with the endplate 6 preventing plunger movement in or out of the barrel. The notches or female fittings 5, which can be on one or more (two in the figure) of the vanes of the plunger, are lined up with the endplate and then rotated 45 degrees to lock the plunger in the example in the figure; the plunger and notches can be rotated an additional 45 degrees in either direction to release the plunger. A luer or other fitting 9 mounts with the syringe barrel, as does a finger operated valve 10 with an appropriate luer 11 or other fitting to join with needles or other compatible medical devices. The valve 10 in the figure is a slide valve with a movable finger rest 12, but many other finger operated valves can be suitable for use with this device. In some applications it can be advantageous to provide a valve that can be operated with the thumb or index finger rather than require the entire hand to operate the valve.

As examples of suitable valves, U.S. Pat. No. 4,819,684 to Zauge et al., U.S. Pat. No. 5,203,769 to Clement et al., U.S. Pat. No. 5,285,805 to Proper, U.S. Pat. No. 5,865,812 to Correia, U.S. Pat. No. 6,972,008 and U.S. Pat. No. 7,198,619 to Bills e al., U.S. Pat. No. 7,226,435 to Darnel, U.S. Pat. No. 6,569,117 to Ziv et al. and U.S. Pat. No. 5,046,528 to Manska disclose examples of valves that can be operated with one finger and can be suitable for use with the present invention. Also, standard or modified turncock and stopcocks can also be used; modifications that permit operation with one finger can provide advantages in some applications.

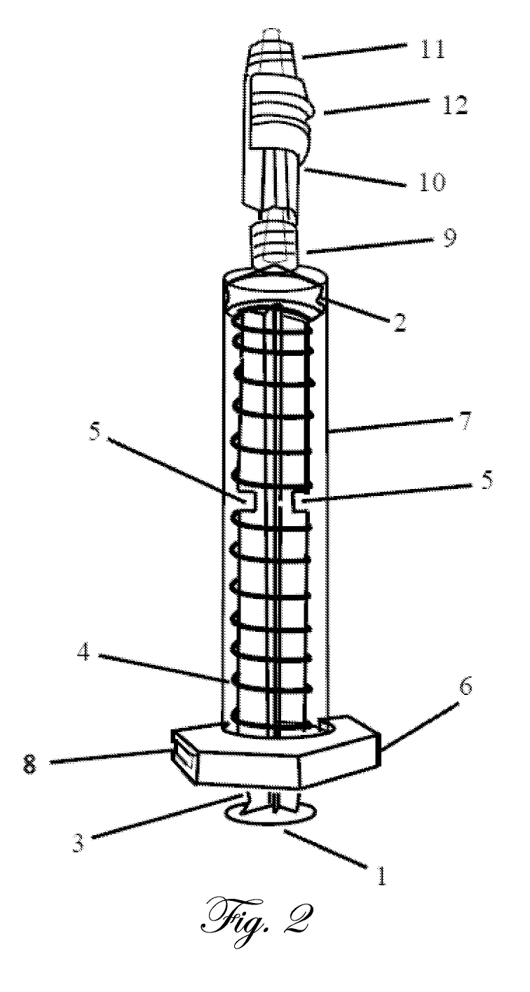

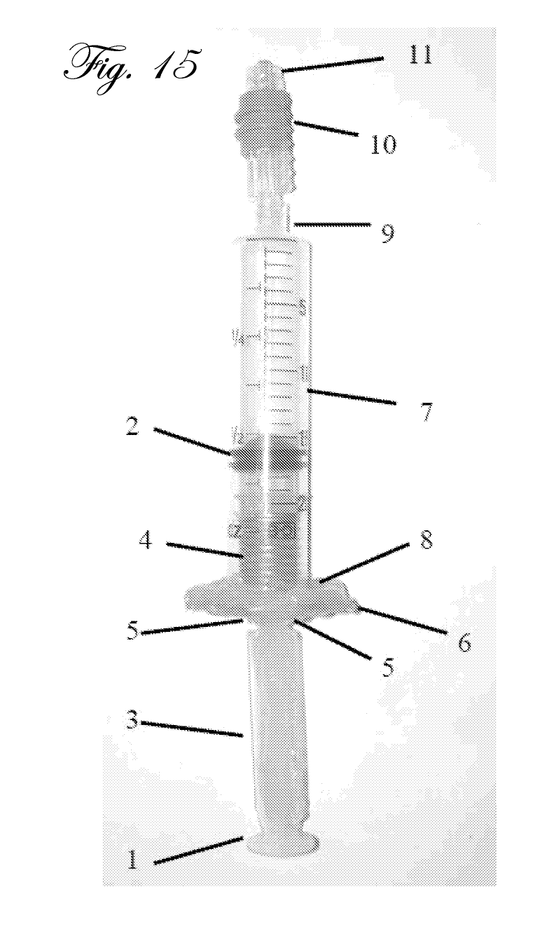

FIG. 2 is a schematic illustration of an assembled automatic syringe. FIG. 15 is a photograph of an example embodiment similar to that described in connection with FIG. 2. To assemble the example automatic syringe, the spring 4 is first placed over the plunger body 3. Since the plunger body 3 is of lesser diameter of than the stopper or seal 2 and usually of the thumb rest or plunger flange 1, the stopper complex 2 or thumb rest 1 can be separate components that fit together after the spring 4 is placed on the plunger body. After the spring 4 is placed on the plunger body 3, the stopper complex 2 can be placed into the end of the barrel 7 and the plunger can be depressed until the stopper complex 2 reaches the end of the barrel 7. At this point the spring is partially compressed and the endplate 6 is placed on or over the finger flanges 8, sealing the spring 4 surrounding the plunger 3 in the barrel. The valve 10 can then be placed or rotated into the needle fitting 9 of the syringe. The automatic syringe is then completely assembled and is in its low energy state.

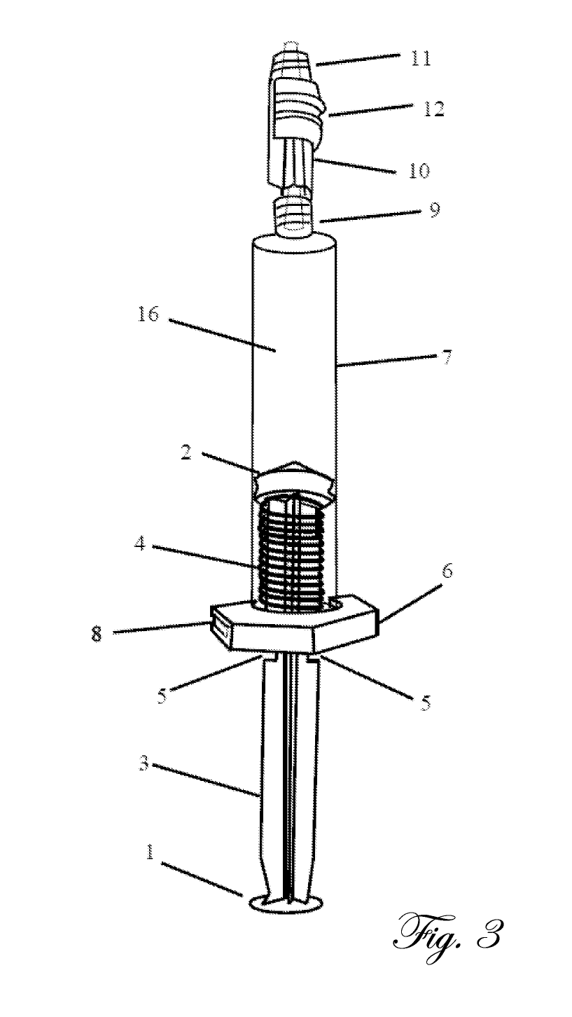

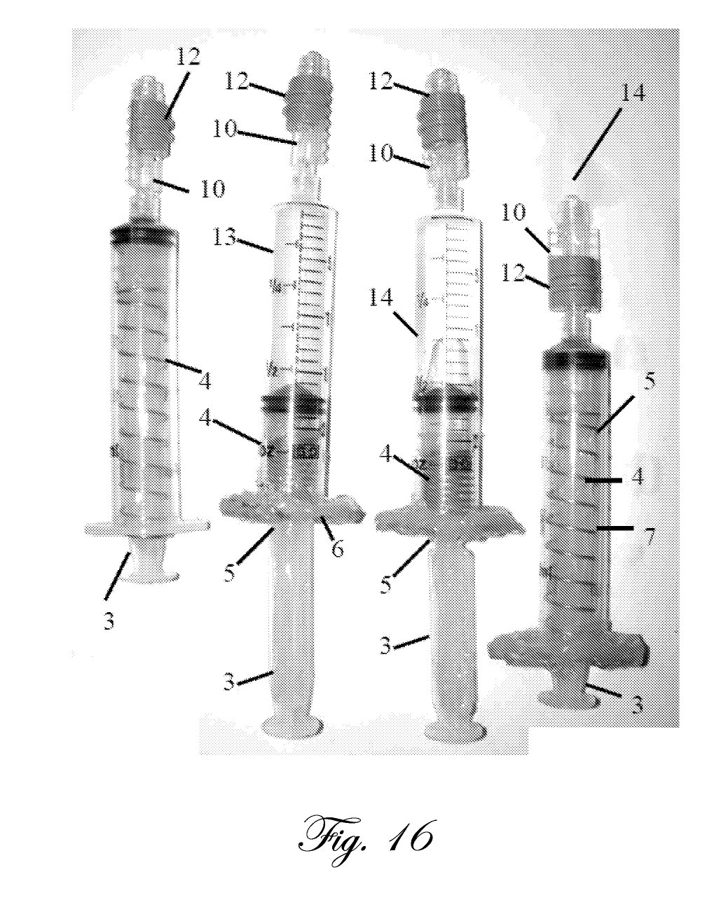

FIG. 3 is a schematic illustration of an assembled automatic syringe ready for use with the valve closed, the spring compressed, and the plunger locked, creating vacuum in the syringe barrel. FIG. 16 is a series of photographs of an example embodiment similar to that described in connection with FIGS. 3-5. In FIG. 3, an assembled automatic syringe is ready for use with the slide finger rest 12 moved forward and the valve 10 closed, the spring 4 compressed, and the plunger locked 3 by means of the slots 5 interacting with and being restrained by the endplate 6, creating vacuum in the syringe barrel. With the valve 10 in the closed position with the valve finger rest 12 in the forward position, the plunger 3 is pulled back, compressing the spring 4. After the plunger is completely pulled back, the plunger is rotated 45 degrees engaging the notch or notches in the plunger with the endplate of the barrel, and locking the plunger in place, creating vacuum 13 in the syringe barrel. The plunger 3 is locked in position by the plunger notches 5 engaging with the endplate 6. In this position, the automatic syringe is now a vacuum syringe that can be used for suction biopsy, aspiration of body fluids, or automatic filling of the syringe with medications.

To perform a suction biopsy such as fine needle aspiration biopsy with the automatic syringe in this position, a needle or other biopsy device is attached to the fitting of valve 10, the needle is directed to the tissue to be biopsied, the valve finger flange 12 is moved to the open position, the vacuum 16 in the barrel is then in communication with the needle, and tissue is forced into the needle by ambient pressure. After the biopsy is performed, the valve flange 12 is moved to the closed position and the needle and syringe removed from the tissues. If the valve 10 is opened at this point, the tissue will be forced into the barrel of the syringe, which is disadvantageous. To prevent this, the vacuum is released by rotating the valve, breaking the airtight seal, and permitting air into the syringe barrel and thus removing the vacuum. The valve 10 is then rotated back to create a seal with the barrel, and then the plunger is rotated 45 degrees disengaging the plunger notch 5 from the endplate 6, permitting the spring 4 to urge the plunger forward creating pressure in the barrel. The needle is then placed above a glass slide or specimen container and the valve is opened, the spring 4 urges the plunger 3 forward into the barrel, and the pressure pushes the tissue sample out of the needle. To take another biopsy, the valve 10 is closed by pushing the valve flange forward to the closed position, the plunger is pulled back, compressing the spring, and the plunger is rotated 45 degrees so that the notches engage 5 engage the endplate 6 again creating and holding a vacuum 13 in the syringe barrel. The automatic syringe is then ready for the next biopsy. Additional details of the clinical use of the device are discussed below.

To aspirate body fluids, the same procedure is performed but the needle tip is placed into a fluid filled body cavity and when the valve is opened the syringe automatically fills with body fluid. To fill the automatic syringe with medication or other liquid, the same procedure is performed, but the needle pierces a stopper or vial, and the needle tip is placed in the medication or other liquid, and the valve is opened filling the syringe with medication or other liquid. Alternatively, the valve 10 without a needle but seated on the automatic syringe can be directed first into a well of medication, saline, or other liquid, and then the valve flange moved to the open position, and the syringe will fill with saline or fluid. Thus, this one automatic syringe in the vacuum position can be used for automatic suction biopsy, aspiration of body fluids, or automatically filling the syringe with saline or medications.

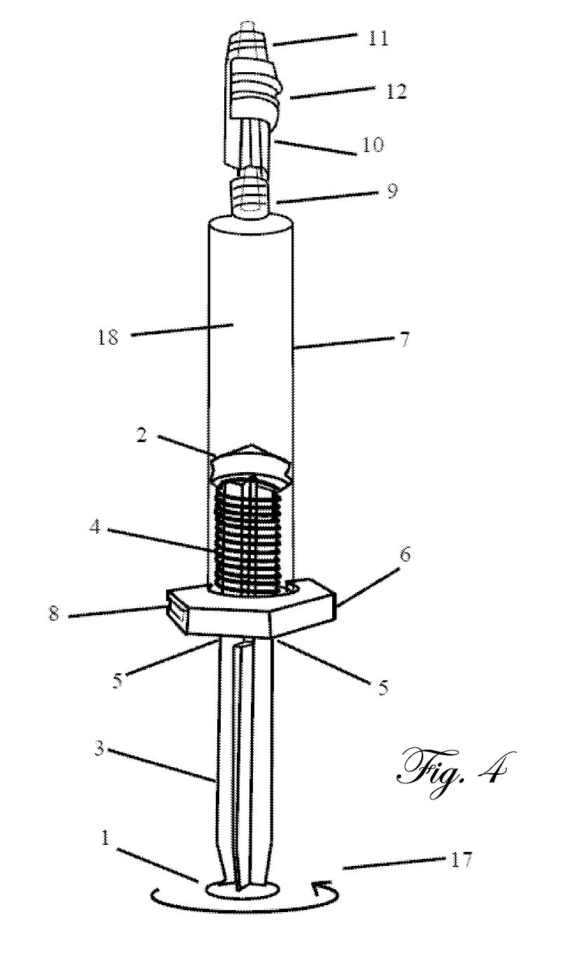

Once the syringe is filled with fluid, medication, or saline, the automatic syringe can be converted from an automatic suction syringe to an automatic injection syringe. As shown in FIG. 4, this can accomplished by moving the valve flange 12 to the closed position, and rotating 17 the plunger 3, disengaging the plunger notches 5 from the end plate 6, permitting the plunger to move and allowing the compressed spring 4 to urge the plunger forward, creating pressure 18 within the barrel 7.

FIG. 4 shows the automatic syringe in a preinjection position with valve 10 closed with the valve flange 12 forward, the spring 4 compressed and urging the stopper and plunger 3 forward, the barrel filled with liquid under pressure 18, the plunger notches 5 disengaged from the endplate, and the plunger 3 unlocked and urged forward by the spring 4 creating pressurized liquid 14 in the syringe barrel. The syringe in the position of FIG. 4 is stable, but is ready to inject once the valve 10 is opened by moving the valve flange 12 to the open position. To automatically inject, the valve flange 12 is moved to the open position, and the pressurized fluid exits the valve 10 through the needle fitting 11 and the plunger 3 moves further forward being urged by the spring 4. The rate of flow of the fluid out the valve can be controlled by the operator by three simultaneous methods 1) finger control of the adjustable valve 10 by moving the valve flange 12 in various positions which controls flow from valve, 2) pre-selection of needle diameter with larger diameters permitting faster flow and smaller diameters enforcing slower flow, and 3) needle length with longer needles enforcing slower flow and short needles permitting faster flow.

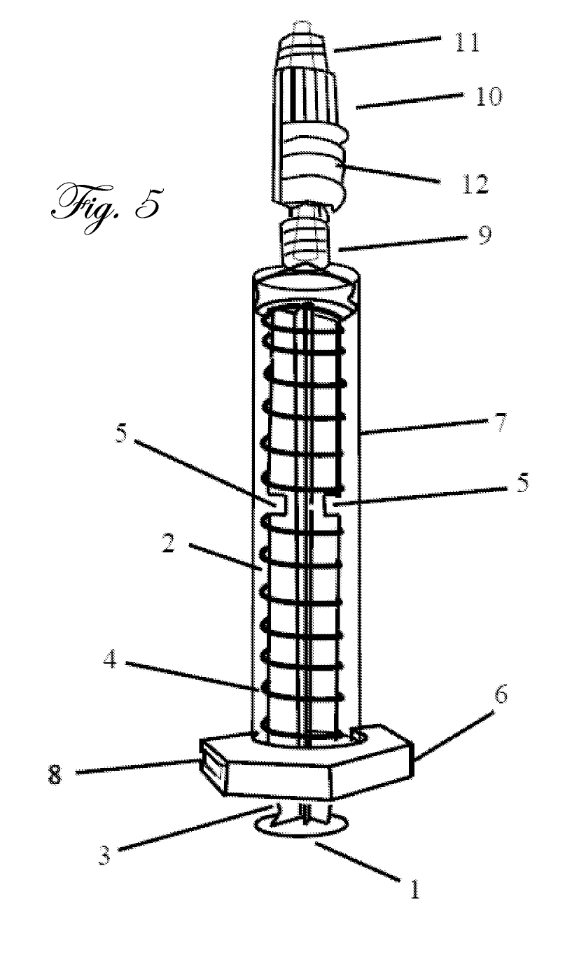

FIG. 5 is a schematic illustration of an automatic syringe with the valve open, the spring uncoiled and urging the stopper forward, the plunger unlocked, and the barrel empty of liquid, it having been expelled out the front of the syringe. FIG. 5 shows the automatic syringe in a post-injection phase with the valve 10 and valve flange 12 in the open position, the spring 4 uncoiled and urging the stopper forward against the forward end of the barrel, the plunger 3 unlocked, plunger slit 5 interior to the barrel, and the barrel 7 empty of liquid, the fluid having been expelled out the needle fitting 11 in front of the syringe.

Example uses for the automatic injection syringe include ultrasound-directed regional nerve blocks, administration of local anesthesia, dilation of the intraarticular space prior to injection of intraarticular medications, ultrasound-directed hydrodissection that is becoming an increasingly used technique in interventional sonography, and location body compartments as would be used for epidural anesthesia and injection of epidural medications.

For use in epidural procedures, the automatic syringe is placed in the vacuum position as in FIG. 4, and then filled with saline by opening the valve 10. After being filled with saline, the valve 10 is closed by moving the valve flange 12 to the closed position. The plunger 3 is then rotated 45 degrees disengaging the plunger notches 5 from the endplate 6, and permitting the plunger to move forward and create pressure within the syringe. The epidural needle is first advanced into the interspinous ligament, the stylet is removed, and the automatic syringe filled with saline attached to the fitting of the needle. The valve is then opened, but the saline has great resistance to exiting with the tough connective tissue of the interspinous and paraspinous ligaments. However, when the loose connective tissue of the epidural space is contacted the resistance to injection rapidly decreases and the automatic syringe injects and the plunge visibly moved. The automatic syringe is then removed from the needle fitting and the medication injected into the epidural space.

To permit this automatic syringe to both automatically aspirate and inject, a mechanism to lock or fix the plunger in position can be provided. In an example embodiment, structures on the plunger reversibly engage structures on the syringe barrel, the reversible engagement and release actuated by rotating the plunger, for example 45 degrees although other degrees of rotation can also function. To accomplish this the plunger body must be of variable dimensions as rotated relative to structures on the barrel that are fixed.

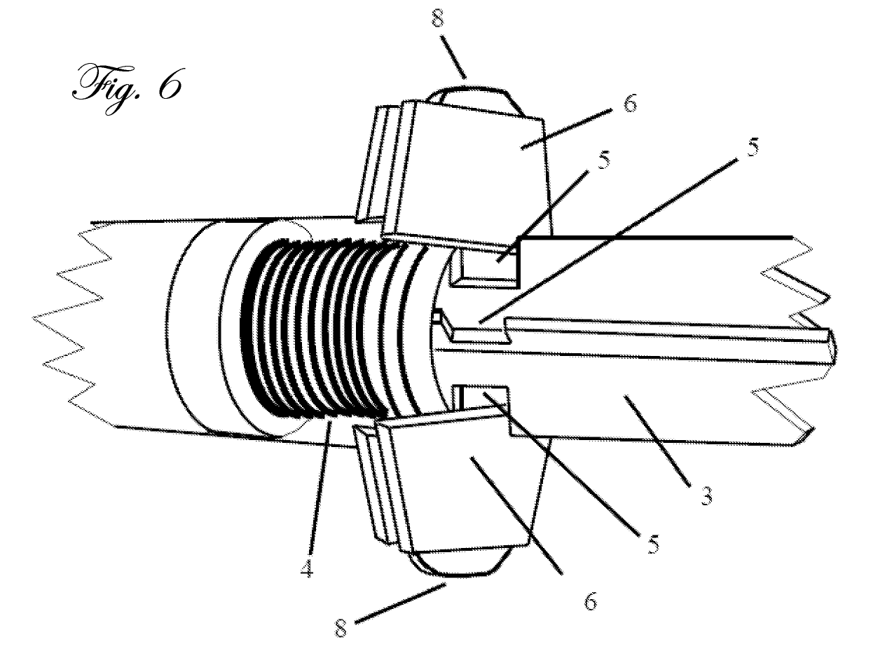

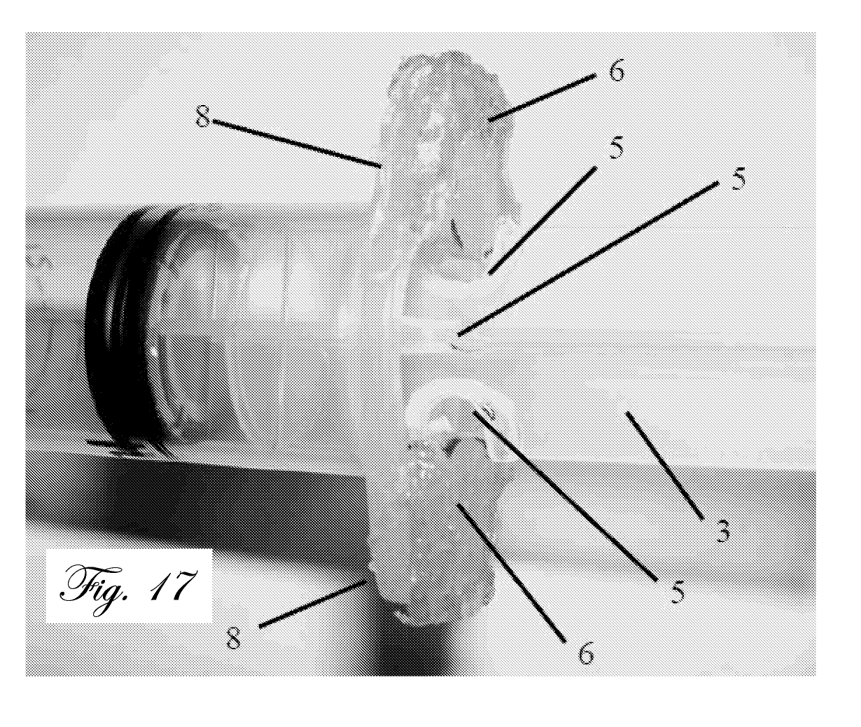

FIG. 6 is a schematic, close-up, illustration of an automatic syringe plunger illustrating the reversible interaction of the plunger notches with the endplate. FIG. 17 is a photograph of an example embodiment similar to that described in connection with FIG. 6. FIG. 6 provides a view of an automatic syringe plunger demonstrating the reversible interaction of the plunger notches 5 with the endplate 6. This type of locking syringe plunger is largely conventional in general structure, except being narrower in order to accommodate the spring 4 but can also be largely conventional plunger without a spring. In the embodiment of FIG. 6, the plunger 3 is of a conventional structure composed of 4 struts or vanes, making the plunger larger in diameter in the right angle position of the struts and diameter shorter when the plunger is rotated 45 degrees. Thus, the endplate 6 which retains the spring 4 within the barrel is sized to fit flush or near flush with the struts or vanes of the plunger when rotated at 45 degrees, permitting the plunger to move freely in or out of the barrel. Into the struts are cut or molded slots or notches 5, that when rotated so that the struts are at right angles to the endplate 6, the notches 5 accommodate the endplate 6 locking the plunger in position making the syringe stable so it does not inject or aspirate without control of the operator. The endplate 6 can be a clip as shown in FIG. 1 and FIG. 2 that snaps over the barrel and finger flanges 8, but can be molded into barrel, or can be welded or glued onto the barrel, but in all forms it can retain the spring within the barrel and can reversibly lock the barrel in position. The endplate can also be a movable structure such as a peg that moves into a corresponding hole or receptacle in plunger. Also, the plunger can comprise other shapes such as ellipsoid in cross-section that vary in diameter when rotated relative to the endplate. Also, the endplate features can be configured to rotate while the plunger remains rotationally stable to provide the required locking function.

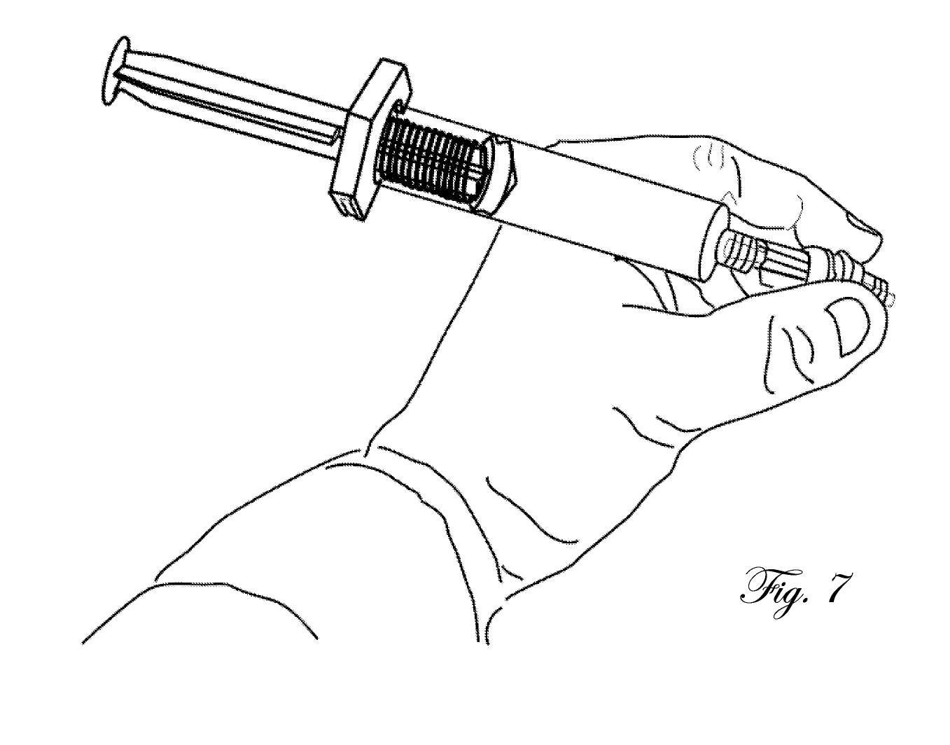

FIG. 7 is a schematic illustration of an automatic syringe held in a one-handed fashion in the pencil grip with the thumb-operated valve. FIG. 7 shows the automatic syringe held in a one-handed fashion in the pencil grip with the thumb-operated valve. This hand positioning can be used for both aspiration and injection with the automatic syringe. The automatic syringe can also be held in other handgrips effectively.

The automatic syringe can be used as well without the valve, although this restricts its uses relative to the valve version. A valveless automatic syringe can be filled directly by pulling back on the plunger while the needle fitting is attached to or submerged in a fluid source. To retain the fluid, the plunger is rotated and locked. To inject the fluid, the plunger is rotated and the spring automatically injects. This valveless automatic syringe can be useful for prefilled syringes and other injection procedures including body space location, especially epidural injections. For epidural location and injections, this syringe can be used similarly to that described by Sundar et al. in US 20070142766. US 20070244446, US 20070232993, and US 20070213674, but unlike the Sundar devices, the automatic syringe can be prefilled with saline, and is stable in locked position until the operator is ready, with further advantages that the injection feature is not activated by rotating the plunger until the syringe is seated fully on the epidural needle, thus greater control is maintained throughout the procedure.

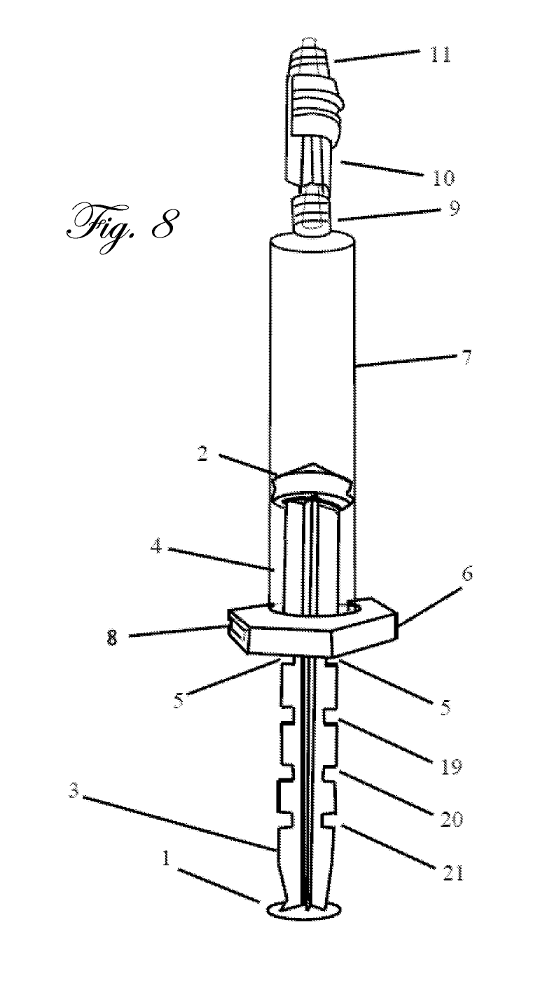

An automatic syringe according to the present invention can also be useful without a spring. FIG. 8 is a schematic illustration of an automatic syringe without a spring. This automatic syringe can be used for aspiration biopsy and for large volume fluid aspiration as demonstrated clinically. This syringe is used in a similar manner to the automatic syringes described in relation to FIG. 1 through FIG. 7 for automatic aspiration, but it does not automatically inject since it does not have a spring, although it can be manually injected. It comprises all the elements of the previously described syringes except for the spring and can have a standard diameter plunger. This example embodiment has a finger operated valve 10, finger flanges 8, endplate 6, plunger notches 5, and a plunger 3 that need not be appreciably smaller than the stopper seal complex since it need not accommodate a spring. The plunger is locked and released by reversible engagement of the plunger notches 5 with the endplate 6 by rotating the plunger as in the previous embodiments. A device such as that shown in the example of FIG. 8 can comprise a 60 ml syringe that can provide various levels of vacuum or aspiration. For example the plunger notch 5 provides vacuum or aspiration equivalent to a 60 conventional syringe, plunger notch 19 equivalent to a 20 ml syringe, plunger notch 20 equivalent to a 10 ml syringe, and plunger notch 21 the equivalent of a 5 ml syringe. Thus, this one device can be used for both an automatic large volume fluid aspiration up to 60 mls or a low vacuum biopsy equivalent to a 5 ml syringe or equivalent to any size syringe in between. These same adaptions can also be used on an automatic syringe with a spring.

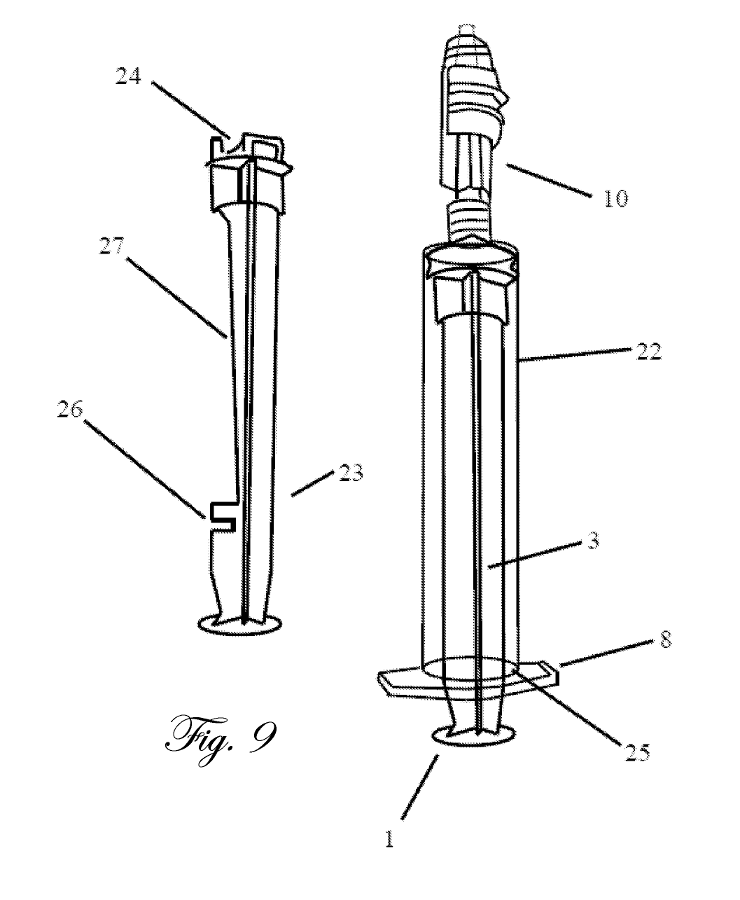

FIG. 9 is a schematic illustration of an example embodiment of an aspiration syringe with a plunger spacer lock. FIG. 9 shows an example embodiment of an automatic syringe with a valve 10, a conventional syringe 22, and a plunger lock 23. This type of plunger lock can be used in any of the other automatic syringes instead of the rotating plunger-endplate engagement system described before. The plunger spacer lock has a forward surface, fitting, or notch 24 that abuts the opening 25 or finger flanges 8 of the barrel, and a rearward surface, fitting, or notch 26 that receives, abuts, mates with, and/or restrains the plunger thumb rest 1. Alternatively, there can be multiple fittings similar to 26 along the plunger lock 23 to receive the thumb rest 1 so that one large syringe could have different levels of vacuum and plunger restraint be volume equivalent to other sizes of syringes as in FIG. 8. Plunger lock 23 can be optimized by have a cut out section 27 that permits smoother movement of the plunger lock over the syringe barrel when pulling the plunger back so that the plunger lock 23 can restrain the plunger 3.

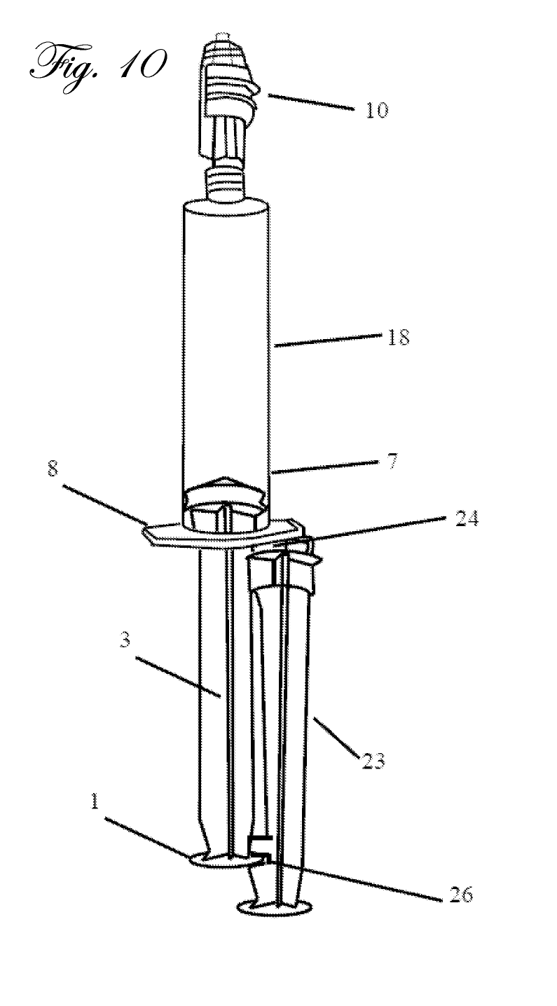

FIG. 10 is a schematic illustration of an example embodiment of an aspiration syringe with plunger spacer lock in place. FIG. 10 shows a plunger lock 23 in place with a lock fitting 24 engaging the barrel 7 at the end or by the finger flanges 8 and the plunger lock fitting or slot 26 accommodating the thumb rest 1 of the plunger 3, creating full vacuum in the syringe because valve 10 is closed prior to pulling the plunger 3 back. To place vacuum in this syringe the valve 10 is closed, the plunger 3 is pulled back, and the plunger spacer lock 23 is fitted in as above. A problem in a large syringe with significant vacuum is the forces on the thumb rest 1 which can fracture the thumb rest as our experiments have shown. To prevent this, the contact area between the slot or lock fitting 26 and the thumb rest 1 can be increased by creating a broad contact area at 26 to distribute the force more evenly over the thumb rest 1.

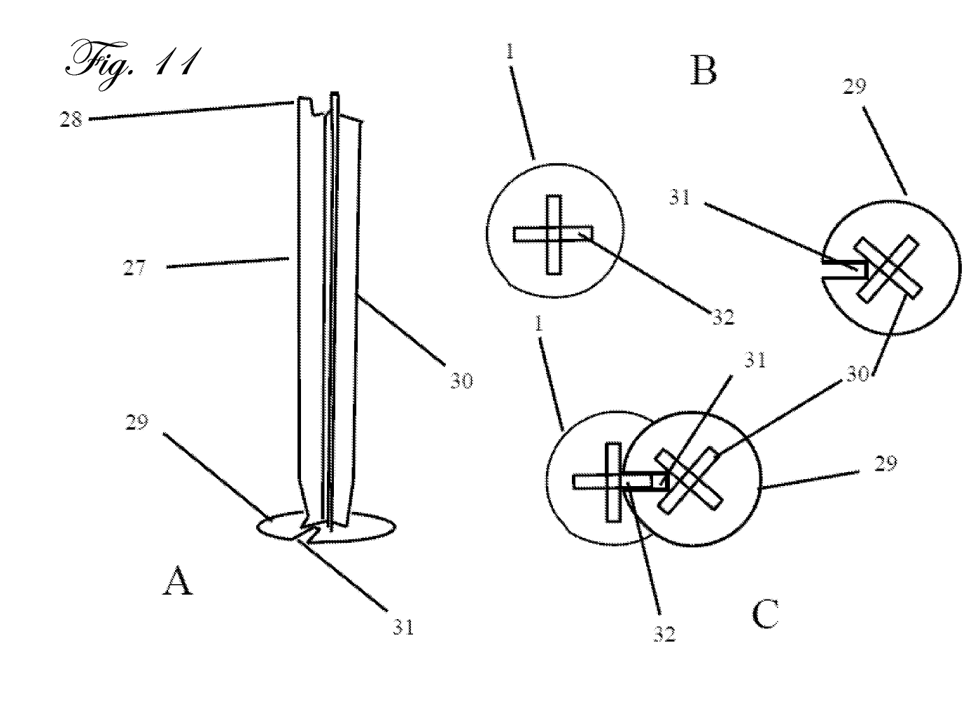

FIG. 11 is a schematic illustration of an example embodiment of a plunger lock. FIG. 11 shows an example embodiment of a plunger lock that distributes the forces evenly over the thumb rest 1. The plunger lock or spacer 27 fulfills the same function as the plunger lock in FIG. 9 and FIG. 10, but has a structure to distribute force on the thumb rest so that it is less likely to fracture when the syringe is in the vacuum phase. As shown in A and B, the plunger lock 27 has a forward surface, fitting, or protrusion 28 to engage the barrel of the syringe, and a rearward surface, fitting, or structure 29 to engage the thumb rest 1 of plunger of the syringe. This example embodiment has a strut or vane structure 30 to provide strength and easy manufacturability, and a slit or female structure 31 to engage a strut or vane 32 of the plunger directly adjacent to the thumb rest 1. C shows the barrel-side of thumbrest of the plunger 1 being engaged on the mating structure 29 of the plunger lock, the female slot 31 engaging the strut or vane 32 of the plunger and the surface area of 29 distributing force more widely over the thumbrest. Since the force is distributed on both sides of the strut 32, it is less likely that the thumbrest 1 will fracture. The struts 30 of the plunger lock do not interfere with the struts 32 of the plunger because they are rotated at 45 degrees to each other.

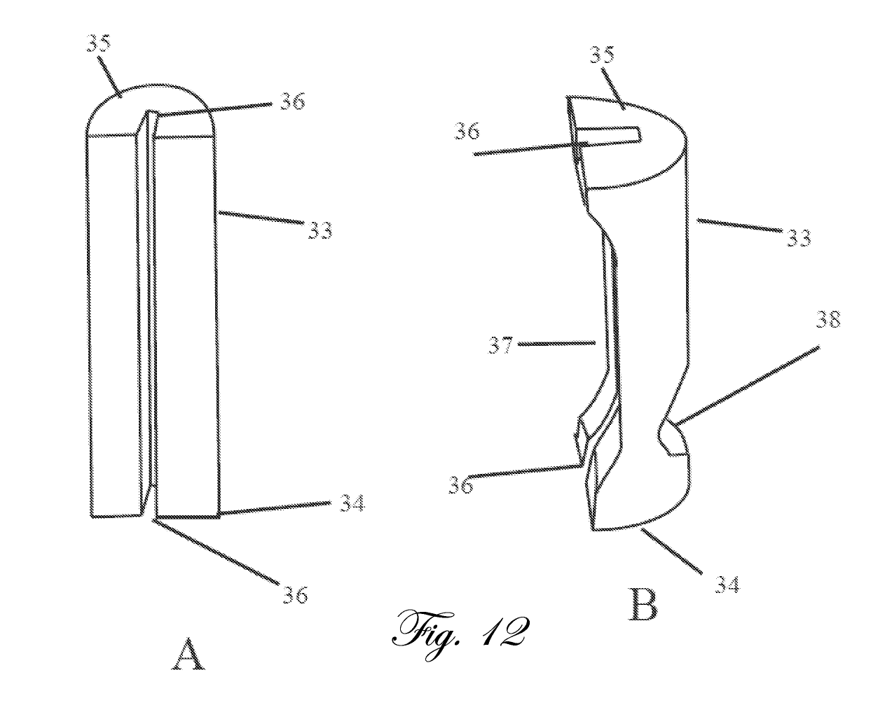

FIG. 12 is a schematic illustration of an example embodiment of a plunger lock. The plunger lock or spacer 33 fulfills the same function as the plunger locks in FIGS. 9, 10, and 11, but has a structure 34 to distribute force on the thumb rest of the plunger so that it is less likely to fracture when a large syringe such as a 60 ml syringe is in the vacuum phase. As shown in A the plunger lock 33 has a forward surface, fitting, or protrusion 35 to engage the barrel of the syringe, and a rearward surface, fitting, or structure 34 to engage the thumb rest of the plunger of the syringe. This example embodiment has a generally solid structure to provide strength and easy manufacturability, and a slit or female structure 36 to engage a strut or vane of the plunger directly adjacent to the thumb rest but potentially along the entire or various parts of its length. Engaging the strut or vane of the plunger prevents the plunger lock from rotating, and thus stabilizes the device when under force. B shows another version of the same embodiment with a cutaway section 37 to permit more facile movement of the device over the syringe barrel when activating and a cutaway finger grip 38 to permit more facile purchase while pulling the plunger back while inserting the plunger lock.

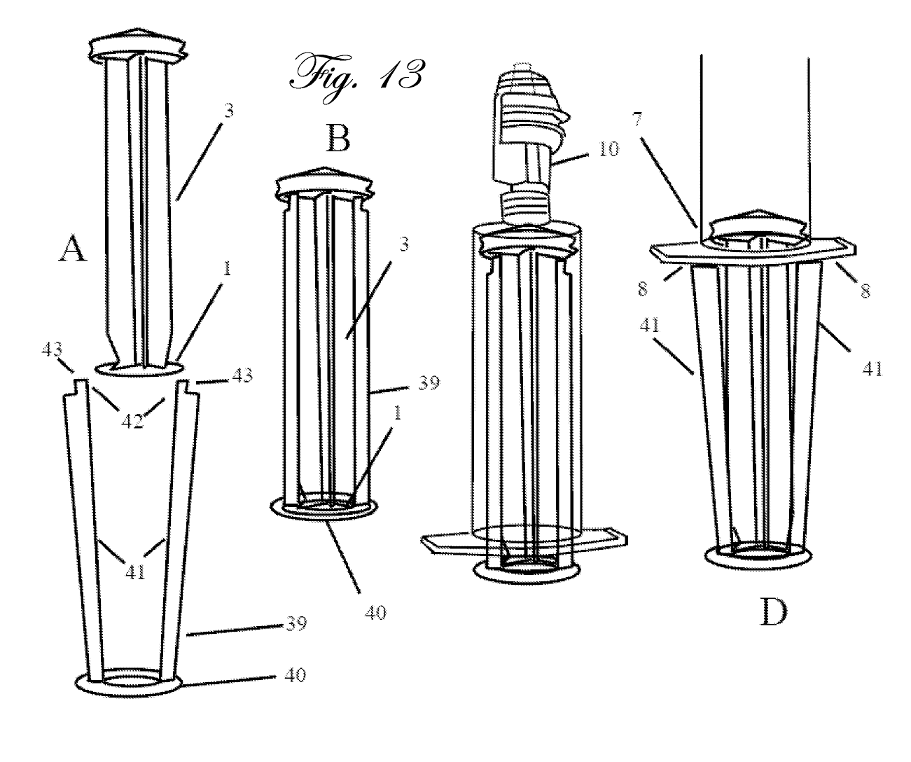

FIG. 13 is a schematic illustration of an example embodiment of a plunger lock. As shown in A the plunger lock or spacer 39 fulfills the same function as the plunger locks in FIGS. 9, 10, and 11, but has a structure 40 to distribute force on the thumb rest of the plunger so that it is less likely to fracture when a large syringe such as a 60 ml syringe is in the vacuum phase. Also, instead of having one solid structure for the plunger lock the example device of FIG. 13 has two or more plunger lock tines 41. As shown in A each of the plunger lock tines 41 have a forward surface or fitting 42 with a protrusion 43 on the inward surface to engage the barrel of the syringe, and a rearward surface, fitting, or structure 40 to engage the thumb rest of the plunger of the syringe. This example embodiment uses two or more plunger lock tines to provide strength, balance and easy manufacturability, and the plunger 3 can be inserted onto or into the plunger lock to convert the plunger into a locking plunger. This plunger lock can also be bonded to the plunger or injection molded as one unitary piece. The tines 41 are angled so that force is required to move them toward the center. B shows the plunger lock 39 engaged with the plunger 3, and the thumbrest 1 of the plunger 3 in direct contact and/or bonded to the distal mating surface 40 of the plunger lock. The tines 41 of the plunger lock have been forcibly moved to the same plane as the plunger, and they reside in the spaces between the struts or vanes of the conventional plunger, but could also reside freely on the outside of a reduced diameter plunger. In any event, with the tines folded in the tines are forced inward and integrated with the plunger so as to have a diameter that permits insertion of both the plunger and the plunger lock into the barrel as shown in C. To create vacuum the valve 10 is placed in the closed position and the plunger combined with the plunger lock is pulled out. As shown in D, when the plunger is pulled out sufficiently the tines 41 recoil towards their original configuration and engage the distal end of the barrel 7 near the finger flanges 8. Because of the structure of the tines 42 with a protuberance 43 (as shown in A), the tines move part way out of the barrel, but are still contained and restrained by the protuberance 43 that remains inside the barrel so that the tines do not disengage the finger flanges of the barrel. The distal surface 40 of the plunger lock 39 directly contacts the thumbrest 1 of the plunger so that force can be transmitted from the plunger 3 and thumbrest 1 to the distal surface 40 of the plunger lock 39 down the tines 41 to the engaging surfaces 42 to the syringe barrel 7. This locks the plunger in the extended position and permits vacuum within the syringe barrel 7. To disengage the plunger lock, the tines 41 are squeezed from outside, forcing them forcing the tines inward towards the center of the plunger 3, reducing the diameter, and permitting both the plunger 3 and the plunger lock 39 to be internalized to the barrel.

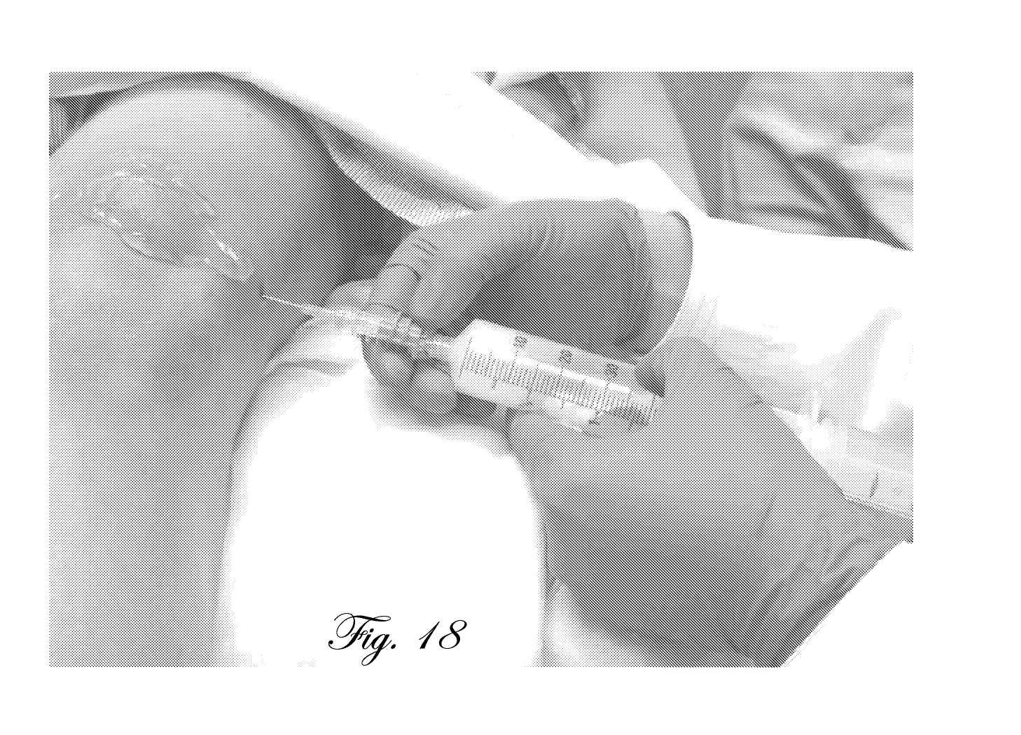

Devices according to the present invention have been tested extensively for control characteristics and hand strength requirements, and the automatic syringes of the present invention are far better controlled and induce less hand fatigue than conventional syringes while affording much improved needle control. FIG. 18 is a photograph of an example embodiment of the present invention in use. Table 1 summarizes some of the benefits attained from use of embodiments of the present invention. As can be seen from Table 1, automatic syringes according to the present invention control the needle significantly better than conventional syringes and are easier for the operator to use.

TABLE 1

| | Conventional | Automatic | | | |
|---|---|---|---|---|---|
| | Syringe (80 | Syringes (80 | | | |
| | procedures | procedures | Percent | Confidence | |
| | each) | each) | difference | Interval | Significance |
| 20 ml Injection | 11.9 ± 4.93 | 5.68 ± 2.93 | −52.30% | −30.4% to −74.1% | P ≦ 0.001 |
| Unintended Forward | | | | | |
| Penetration (mm) | | | | | |
| 20 ml Injection | 4.26 ± 1.85 | 1.84 ± 1.46 | −56.80% | −31.7% to −81.9% | P ≦ 0.001 |
| Unintended | | | | | |
| Retraction (mm) | | | | | |
| 20 ml Injection Ease | 6.28 ± 0.75 | 9.07 ± 1.38 | 36.50% | +38.9% to +49.2% | P ≦ 0.001 |
| of Procedure 0 = very | | | | | |
| difficult 10 = | | | | | |
| extremely easy | | | | | |
| 20 ml Aspiration | 15.8 ± 4.50 | 8.94 ± 4.37 | −43.30% | −25.4% to −61.4% | P ≦ 0.001 |
| Unintended | | | | | |
| Penetration (mm) | | | | | |
| 20 ml Aspiration | 4.89 ± 2.84 | 2.11 ± 1.63 | −56.90% | −26.6% to −87.1% | P ≦ 0.001 |
| Unintended | | | | | |
| Retraction | | | | | |
| 20 ml Aspiration Ease | 5.75 ± 1.33 | 8.57 ± 0.95 | 32.90% | +28.7% to +38.3% | P ≦ 0.001 |
| of Procedure 0 = very | | | | | |
| difficult 10 = | | | | | |
| extremely easy | | | | | |

CLINICAL RESULTS. The following sections present information relating to clinical results using example embodiments of the present invention. The descriptions of the embodiments and their uses are examples and illustrative only, and are not intended to limit the invention.

Fat Biopsy for Amyloidosis. Subcutaneous fat biopsy by needle aspiration is useful for the evaluation of amyloidosis, environmental contaminants, lipid metabolism, genetic studies, and diabetes research; however, aspiration with conventional syringes is awkward and can result in needle trauma to patient tissues. The present study examined new technologies for needle aspiration biopsy.

Materials and Methods: Subcutaneous fat biopsy in 10 high-risk individuals with arthralgias and neuropathic symptoms was randomized to 1) a 10 ml RPD (reciprocating procedure device) mechanical syringe, or 2) a 60 ml vacuum syringe. In each case an 18 gauge 2 inch (5 cm) Chiba biopsy needle was utilized. Outcome measures included patient pain by the 10 cm Visual Analogue Pain Scale (VAS), adequacy of biopsied tissue, complications, and diagnosis. The operator's ability to control the syringe was quantitatively measured by the linear displacement method.

Results: Both the vacuum and mechanical syringes permitted facile aspiration of subcutaneous fat; in all cases, there was adequate sample obtained for analysis without complications. The mechanical and the vacuum syringes enhanced control of the needle compared to conventional syringes, reducing unintended forward penetration by 75% (3.6±0.5 mm) and 87% (12.0±1.4 mm), respectively (p<0.0001). Although adipose cells were obtained in abundance, small blood vessels and connective tissue septa were also obtained intact permitting precise microhistological examination. One case of primary AL amyloidosis (kappa light chain disease) was diagnosed in each group.

Conclusions: Subcutaneous fat biopsy by needle aspiration can be facilely achieved with new aspiration syringe technologies with improved needle control and enhanced patient safety.

Subcutaneous fat biopsy performed with needle aspiration is a standard methodology to obtain adipose tissue for histologic, metabolic, immunological, and biochemical analysis. Subcutaneous fat biopsy has become the standard for the diagnosis of systemic amyloidosis, for metabolic, immunologic, endocrine, and genetic studies of adipose tissue, and for measuring lipophilic environmental contaminants. Most practioners use a 10 or 20 ml syringe, a 16 to 18 large bore needle, and provide vacuum by pulling back the plunger on the syringe to perform multiple passes until enough sample has been obtained. The original description of subcutaneous fat biopsy utilized a 50 ml syringe, which generated considerable vacuum. However, larger syringes are difficult to both operate and control because of the considerable force required to retract the plunger, and can result in needle penetration of the abdominal wall with subsequent hematoma formation.

New technologies, including mechanical syringes and vacuum syringes, have been developed to perform needle aspiration biopsies, but to date these devices have not been used routinely in needle biopsy of subcutaneous fat. Mechanical syringes have proven superior to and safer than the conventional syringe for aspiration procedures. Vacuum syringes >50 ml are used for amniocentesis, suction biopsy, suction curettage, and cosmetic liposuction, but there few reports of the use of large vacuum syringes for fat biopsy. We hypothesized that mechanical syringes and vacuum syringes would provide enhanced needle control for aspiration biopsy of subcutaneous fat. The present trial determined the control characteristics of mechanical and vacuum syringes relative to conventional syringe, and compared fat biopsy performed with a mechanical syringe to that with a vacuum syringe.

Subjects: This project was in compliance with the Helsinki Declaration, approved by the institutional review board (IRB), and was registered at ClinicalTrials.gov (Clinical Trial Identifier NCT00651625). Inclusion criteria included: 1) unexplained arthralgias and neuropathic symptoms consistent with the diagnosis of systemic amyloidosis, 2) negative serological evaluations for the usual causes of these symptomatologies, 3) indication for fat biopsy as determined by an expert physician, and 4) formal consent of the patient to undergo the procedure and participate in the research. A total of 10 individuals with unexplained arthralgias and neuropathic symptoms were randomized between 1) a 10 ml mechanical syringe (5 individuals), or 2) a 60 ml vacuum syringe (5 individuals). Costs for fat biopsy were determined by using HPCP/CPT code 10021 as determined by the US Department of Health & Human Services, Centers for Medicare & Medicaide Services. Patient outcomes included 1) procedural pain as measured by the 0-10 cm Visual Analogue Pain Scale (10 cm VAS), 2) adequacy of the fat biopsy specimen for histopathologic analysis, 3) the diagnosis of amyloidosis, and 4) complications, including bruising, hematoma, or infection.

The abdominal fat biopsy technique was generally similar to that described previously. After informed consent, the area of skin for biopsy on the lateral side of the abdomen was prepared with chlorhexidine antisepsis. Subsequently local anesthesia with 1% lidocaine (Xylocalne® 1%, AstraZeneca Pharmaceuticals LP, 1800 Concord Pike, P.O. Box 15437, Wilmington, Del., USA 19850-5437) was administered intradermally using a 25 gauge ⅝ inch (1.6 cm) needle on a 5 ml mechanical syringe. As in the original description of abdominal fat biopsy by Hirsch et al, a biopsy needle with a stylet was used (Chiba Biopsy Needle 18 gauge 5 cm (2 inch), GPN G02585, ON DCHN-18-5.0, Cook Medical, Bloomington, Ind., USA). The Chiba needle with the stylet was grasped at the front part of the needle between the index finger and thumb with the hub portion resting in the palm. The abdominal skin and fat were then pinched between the fingers and lifted away from the abdominal wall to prevent accidental penetration of the rectus muscles during needle insertion. The skin was then penetrated and the biopsy needle advanced about 1 inch (2.5 cm) into subcutaneous fat. At that point, the stylet was removed and the aspiration syringe was attached, either the mechanical syringe or the vacuum syringe, by either rotating the needle or rotating the syringe into the needle fitting.

Needle Aspiration Biopsy with the Mechanical Syringe. The mechanical syringe was a 10 ml RPD mechanical syringe (RPD 10 ml syringe, AVANCA Medical Devices, Inc, Albuquerque, N. Mex., USA. website: www.AVANCAMedical.com). The RPD mechanical syringe is formed around the core of a conventional syringe barrel and plunger, but has a parallel accessory plunger and an accessory barrel to control the motion of the accessory plunger. The two plungers are mechanically linked by a pulley in an opposing fashion, resulting in a set of reciprocating plungers. Thus, when the injection plunger is depressed with thumb, the syringe aspirates, and when the aspiration plunger is depressed with the thumb, the syringe injects. This permits the index and middle fingers to remain in one position during both aspiration and injection, while the thumb only needs to move in a horizontal plane to the alternative plunger in order to change the direction of aspiration or injection.

Prior to use, the 10 ml mechanical syringe was first cycled to break the bond of the plunger stopper with the barrel, and the air was then expelled by depressing the injection plunger. The mechanical syringe was then rotated onto the indwelling needle (or the needle rotated onto the syringe) and then the aspiration plunger was depressed fully, generating 560 Torr (mm Hg) of vacuum. The fat biopsy was then performed in a conventional manner while depressing the aspiration plunger. After the biopsy was performed, the aspiration plunger was released, releasing vacuum in the syringe. The needle was then extracted. If the sample was small and still within the needle, the needle was removed and air aspirated into the mechanical syringe by depressing the aspiration plunger. The needle was then reattached, and the injection plunger was depressed and the sample expelled onto a slide or into cytological processing fluid. The biopsy process was then repeated. If the syringe were filled with tissue and no further biopsies were necessary, the fat was washed out or removed from the barrel in a conventional manner.

Suction Biopsy with the Vacuum Syringe. The vacuum syringe consisted of a conventional 60 ml syringe (BD 60 ml Syringe, Luer-Lok Tip, REF 309653, BD Franklin Lakes, N.J. 07417). This 60 ml conventional syringe was fitted with a sterile flow switch (QOSINA, Flow Control Switch, part number 97337, Quosina Corp., 150-Q Executive Drive, Edgewood, N.Y. 11717-8329 U.S.A), and a custom plunger lock (60 ml plunger lock, AVANCA Medical Devices, Inc, Albuquerque, N. Mex., USA. website: www.AVANCAMedical.com). To create vacuum in the 60 ml conventional syringe, the flow switch was closed, the distal portion of the plunger lock fitted into the thumb rest of the plunger, the plunger pulled back to the 60 ml mark, and the proximal portion of the plunger lock fitted into the syringe barrel so that the plunger remained at the 60 ml plunger displacement mark. This 60 ml conventional vacuum syringe achieved a vacuum level of 720 Torr (mm Hg).

The 60 ml vacuum syringe was rotated onto the 18 gauge indwelling biopsy needle (or the needle rotated onto the syringe). The flow switch was then opened and the biopsy procedure performed as is fully conventional with multiple passes. Once done with the aspiration procedure, the flow switch was closed and the needle extracted; the closed flow switch prevented air and the sample in the biopsy needle from being sucked into the syringe. To expel a sample in the needle, the flow switch was rotated and loosened to permit air to flow into the syringe barrel, and then rotated in the opposite direction to become airtight again. The plunger lock was removed, and the flow switch opened, and then the plunger was depressed, expelling the sample. If the syringe were filled with fat and no further biopsies were necessary, the fat was washed out or removed from the barrel in a conventional manner.

Processing Cytological Specimens. After expelling the biopsy tissues from the needle and barrel, identifiable tissues were fixed in formalin and embedded in paraffin as previously described, then stained with a freshly prepared solution of alkaline Congo Red and examined under polarizing microscopy at a magnification of ×10 and ×100 to assess the presence of the light pink material typical of amyloid deposits on light microscopy and the focal apple green birefringence of amyloid deposits on polarizing microscopy. For biopsies without identifiable tissues, the cytologic sample was centrifuged, formalin fixed, and the cellular plug embedded in paraffin and stained with Congo Red and similarly accessed for the presence of amyloid. After demonstrating amyloid deposits, the clinical diagnosis of amyloidosis was confirmed by serum immunoelectrophoesis and measurement of circulating light chains as appropriate.

Quantifying Syringe and Needle Control During Fat Biopsy Procedures. The validated quantitative needle-based displacement method was used to measure needle control in conventional, mechanical, and vacuum syringes. This method measures loss of control in the forward direction (unintended forward penetration) which has been associated with needle trauma, hemorrhage and increased pain in syringe procedures. A rigid polystyrene marker is placed on the needle to a preset indelible mark on the needle, and then the needle is advanced into the target until the polystyrene marker was touching the surface of the target tissue. The operator then performs the biopsy syringe procedure. Loss of control in the forward direction (unintended forward penetration) pushes the polystyrene marker posteriorly on the needle past the indelible mark, permitting precise measurement in mm of loss of control. The needle is marked every 10 mm, which allows precise measurement of loss of control in either the forward or reverse direction. For recording measurements less than 10 mm, a standard metric caliper is used. Finally, the operator recorded the difficulty of each procedure with each syringe using the visual analog scale for difficulty (10 cm VAS). Using the 10 cm VAS, 0 cm=no difficulty and 10 cm=extreme difficulty.

Control of the 10 ml mechanical fat biopsy syringe was compared to control of 10 ml conventional syringe, control of the 60 ml vacuum syringe was compared to the 60 ml conventional syringe. Aspiration procedures were performed for 10 cycles each with a conventional syringe and the mechanical and vacuum syringes and mean and standard deviation calculated. The order of each procedure with each syringe was randomized to reduce the possibility of a consistent bias or a bias from a training effect.

Statistical Analysis: Data were entered into Excel (Version 5, Microsoft, Seattle, Wash.), and analyzed in SAS (SAS/STAT Software, Release 6.11, Cary, N.C.). Differences between parametric two group data were determined with the t-test. Differences in categorical data were determined with Fisher's Exact Test, while differences between multiple parametric data sets were determined with Fishers Least Significant Difference Method. Corrections were made for multiple comparisons. Correlations between parametric data were determined with logistic regression and between non-parametric data with Spearman correlation and Kendall rank method.

Results: Both aspirating syringe technologies were better controlled and easier to operate compared to the corresponding conventional syringe. The 10 mechanical syringe reduced unintended forward penetration of the needle tip during aspiration by 75% (3.6±0.5 mm) (p<0.0001), and reduced the difficulty of aspiration by 64% (4.9±0.5 cm) (p<0.0001). The vacuum syringe was also superior to the conventional syringe, reducing unintended forward penetration of the needle tip during aspiration by 87% (12.0±1.4 mm), and reduced the difficulty of operating the syringe by 74% (7.2±0.5 cm) (p<0.0001). Thus, both aspirating syringe technologies, the vacuum syringe and RPD mechanical syringe, were superior to the corresponding conventional syringe in needle control characteristics and ease of use.

Direct clinical comparisons between the mechanical syringe and vacuum syringe were made. Both technologies permitted facile fat aspiration, and were equivalent in procedural pain, adequacy of the sample, and clinical diagnosis. Complications including an incidence of minor bruising in the vicinity of the puncture site occurred in 20% of the individuals, but no infections and no hematoma.

Although providing considerable fat droplets and septal debris as previously described in aspiration fat biopsy, the Chiba cutting biopsy needle also provided intact columns of tissue including fat septa and vascular elements that permitted precise anatomical structure and facilitated both Congo Red and polarizing microscope examinations. One individual in each group demonstrated typical amyloid deposits, and after further evaluation, was diagnosed with primary AL amyloidosis confirmed by biopsy of bone marrow and/or bowel and with serum confirmation of elevated light chains.

Discussion: The present study demonstrates that the mechanical syringe and the vacuum syringe are better controlled and are easier to operate than a conventional syringe for fat aspiration biopsy. Further, the study demonstrates that biopsy is facilely achieved with the vacuum syringe and the mechanical syringe with equivalent outcomes including procedural pain, adequacy of tissue, and clinical diagnosis. Although fat droplets, individual adipose cells, and septal fragments are obtained as in other biopsy studies, certain subcutaneous fat biopsy samples obtained with the mechanical syringe and vacuum syringe, because of the cutting Chiba biopsy needle and higher levels vacuum, retained the native architecture with less fragmentation typical with conventional fat biopsy methods. The typical needles conventionally used for fat biopsy are conventional non-coring needles, where the surface of the bevel of the needle is designed to move tissue to the outside of needle and not enter the bore. Thus, in most fat biopsies, the tissue must be macerated by the multiple thrusts of the needle breaking the septa and freeing the fat cells so that the tissue becomes a suspension and can be aspirated into the needle. Although the same mechanical breaking occurs with the Chiba cutting needle providing a suspension of fat cells and fragmented septa, columnar biopsies including intact blood vessels and septa are also obtained permitting precise histological staining and examination for amyloid deposits. Of course, standard and non-coring needles could also be used with the vacuum and mechanical aspirating syringes.

Subcutaneous fat biopsy performed with needle aspiration has become a routine methodology in rheumatology, neurology, environmental medicine, nephrology, and endocrinology to obtain adipose tissue for histologic, metabolic, immunological, and biochemical analysis. Subcutaneous fat biopsy has become the standard for the diagnosis of systemic amyloidosis, for metabolic, immunologic, endocrine, and genetic studies of adipose tissue, and for measuring lipophilic environmental contaminants especially pesticides and halogenated hydrocarbons. Most practioners use a 16 to 18 large bore needle, and provide vacuum by pulling back the plunger on the syringe, and performing multiple passes until enough sample has been obtained. Typically, as described by Hazenberg et al, Westermark et al, and Daum et al a 10 to 33 ml syringe is utilized to provide vacuum. However, the original description of needle biopsy of fat by Hirsch et al in 1960 describes the use of the larger 50 ml syringe, which generates considerable vacuum to promote increased biopsy yield. However, larger syringes are difficult to operate and control because of the force required to extract the plunger, and can result in unintended forward penetration of the needle with injury to the abdominal musculature or blood vessels with subsequent hematoma formation. Even a 10 ml syringe can be dangerous when aspirating, resulting in loss of control of up to 6 mm, resulting in perforation or hemorrhage. This is especially a problem in a slender individual with little abdominal fat, and consequently the skin and subcutaneous tissues must be grasped and lifted off of the abdominal wall so that unintended puncture of the abdominal musculature or wall does not occur, particularly in a novice operator's hands.

A number of devices have been developed to improve vacuum assisted needle biopsy, including syringe handles, plunger locks, syringe pistols syringe guns, three-ringed syringes, and mechanical syringes. Of these various devices, mechanical syringes have proven to be the best controlled of devices during aspiration, and have the advantage that these devices can both facilely aspirate and inject. Mechanical syringes have a reciprocating mechanism that prevents device lengthening during aspiration-injection, and permit improved needle control and aspiration-injection characteristics. Clinical trials have demonstrated that mechanical syringes are superior for aspiration procedures to a conventional syringe, resulting in greater needle control, less procedural pain, greater sample yield, less needle trauma, and a reduced incidence of hemorrhage. Mechanical syringes also provide greater safety and improved outcomes for other needle procedures, including thyroid aspiration procedures, suction biopsy, abscess aspiration, administration of local anesthesia, sclerotherapy, central venous access, breast procedures, and ultrasound-guided procedures. The present study confirms that mechanical syringes are superior in control characteristics to conventional syringes, reducing the dangerous unintended forward penetration by 75% during an aspiration procedure, and further demonstrates that effective subcutaneous fat biopsy can be facilely performed with these devices. Mechanical syringes cost approximately $2 US amounting to 2% of the total cost of a fat biopsy and because of improved outcomes and safety, are considered cost-effective.

Westermark et al described the use of a vacuum syringe for fat biopsy consisting of a needle cap as a plunger lock on a 10 ml syringe (“Tarek's trick”) for fat biopsy, but this maneuver is not adequate for larger syringes, which require a longer, more robust plunger locks and, because of the considerable forces required to generate vacuum in a large syringe, are more dangerous in terms of needle control in situ while the needle tip is close to abdominal wall. Dedicated large-volume vacuum syringes have been used for amniocentesis, liposuction, vacuum curettage, and fine-needle aspiration biopsy. Certain these syringes are both reusable and expensive in the range of $100 per syringe. The expense of these dedicated vacuum syringes are related to the low volume of production, the use of non-standard non-conventional syringe components, the requirement that the devices be sturdy during the aspiration procedure and not permit flexing, bending, or dislocation at the needle fitting, and the presence of non-standard non-luer fittings. All of these characteristics make dedicated vacuum syringes unsuitable for diagnostic fat biopsy that must utilize inexpensive technologies to be cost-effective.

In the present study, to generate high levels of vacuum yet keep costs low, a conventional 60 ml syringe out of the package (approximately $1.00 US per syringe) was used for the vacuum syringe. A variable control on-off flow valve (approximately $0.50 US per valve), and a custom plunger lock (estimate $0.50 per plunger lock) were then attached to permit precise release of vacuum and generation of vacuum, respectively. This type of simple vacuum syringe increases the cost of fat biopsy by a net of $1.00 US, approximately 2% of the total costs of a fat biopsy procedure, yet permit one-handed operation, high levels of vacuum, and exquisite needle control. Alternative designs of plunger locks that require special integrated parts, that are internalized into the syringe barrel, require changes in the design of the plunger, or require assembly before the syringe is packaged all could have functional advantages, but have a tendency to increase costs due to low-volume production, the need for new precise molds, additional assembly costs, and the inability to use low-cost off-the-shelf sterile syringes. The type of vacuum syringe with an integrated plunger lock would make sense if the same vacuum syringe technology were used frequently in other aspiration procedures (amniocentesis, thoracentesis, paracentesis, arthrocentesis, fine needle aspiration biopsy, etc) so that the volume of production would lower costs; for fat biopsy alone, as shown here, the use of a off-the-shelf sterile conventional 60 ml syringe with simple application of an external on-off valve and plunger lock is probably the most cost-effective approach.

The present study demonstrates that vacuum and mechanical syringes are superior in control characteristics to conventional syringes, reducing the dangerous unintended forward penetration by 87% (12.0±1.4 mm) in vacuum syringes and 87% (4.9±0.5 cm) in mechanical syringes during an aspiration procedure. The study also demonstrates that the vacuum syringe provides outcomes equivalent to that obtained with the highly controllable mechanical syringe, a syringe technology that has proven to improve outcomes of syringe procedures relative to conventional syringes.

In summary, fat biopsy can be facilely achieved with new syringe technologies, including mechanical syringes and vacuum syringes and provide improved needle control, one-handed operation, and enhanced patient safety.

Automatic Syringes and Sonography. The present study investigated the needle control characteristics of a new automatic syringe that can both automatically aspirate and automatic inject while held in the pencil grip during image-guided procedures.

Methods: The 10 ml and 20 ml automatic syringes were compared to the corresponding conventional syringe during aspiration, suction biopsy, and injection procedures. The validated quantitative needle-based linear displacement method was used to measure control of the syringe and needle. Difficulty of performing each procedure was measured with the 10 cm Visual Analogue Difficulty Scale (VAS).

Results: Compared to the corresponding conventional syringe, the 10 ml and 20 ml automatic syringes reduced dangerous unintended forward penetration of the needle tip during 1) fluid aspiration by 73% (−3.1±0.6 mm) and 75% (−3.6±0.5 mm), respectively (each p<0.001), 2) aspiration biopsy by 71% (−2.9±0.6 mm) and 72% (−3.3 mm), respectively (each p<0.001), and 3) controlled injection by 71% (−3.3±0.6 mm) and 67% (−2.9±0.6 mm), respectively (each p<0.001). The automatic syringe consistently reduced the difficulty of each procedure by 61% to 77% (p<0.001).

Conclusions: Automatic syringes are significantly better controlled and are less difficult to operate than conventional syringes during image-guided aspiration and injection procedures.

Ultrasound-directed needle and syringe procedures, originally pioneered by interventional radiologists and obstetricians, are increasingly used throughout medicine, including critical care, emergency medicine, anesthesiology, nephrology, endocrinology, and the musculoskeletal clinic amongst others. Presently, there are two basic operator methods for using ultrasound to direct needle placement into an anatomic target. In the first method, the operator guides the syringe and needle while an ultrasound technician or assistant controls the ultrasound probe. An alternative method has the operator guiding the syringe and needle while the same operator also controls the ultrasound probe. Although radiologists typically have an ultrasound technician to assist them with the procedure, other specialists who use sonography often do not have the immediate assistance of an ultrasound assistant, and thus operate both the ultrasound probe and syringe themselves.

Although most modern ultrasound devices have the capabilities of measuring target depth and angle from the transducer and these measures markedly assist in proper initial placement of the needle tip, many sonographic procedures including anatomic hydrodissection, nerve blocks, epidural space detection, suction biopsy, injection of tight joints such as the hip, and aspiration of collapsing cystic or multicystic structures, require frequent real time adjustment of needle tip position or rotation of the bevel under fine control while simultaneously aspirating or injecting with a syringe, maneuvers that can be difficult with one hand. Because of this, technologies that permit one-handed use and fine control of a syringe while aspirating and injecting so that the free hand can operate the ultrasound probe have assumed greater importance.

We hypothesized that an automatic syringe that could be held in the finger grip positioning would be better controlled than a conventional syringe during aspiration and injection procedures. In the present randomized, controlled study, we compared the control and performance characteristics of a new automatic syringe with a conventional syringe in image-guided aspiration and injection procedures.

Procedure Syringes: The conventional syringes used for comparative testing were the 10 ml and 20 ml Luer-Lok BD syringes (Ref U.S. Pat. No. 309,604, 309,661, Becton Dickinson, Franklin Lakes, N.J., USA). The automatic syringes were the 10 ml and 20 ml prototypes consisting of 1) a conventional syringe core, 2) a flow control switch to adjust fluid flow and to permit either injection or aspiration, 3) an internal spring enclosed in the barrel to provide pressure and injection capabilities, and 4) a plunger accommodating the spring and possessing a locking mechanism to permit automatic filling and to provide vacuum and aspiration capabilities.

To prepare the syringe for automatic aspiration for suction biopsy or aspiration of body fluid, the control switch is placed in the off position, the plunger is pulled back to create vacuum in the syringe and to simultaneously compress the spring, and the plunger is rotated to lock the plunger in the aspiration position. To aspirate body fluid or to perform a suction biopsy, the procedure needle is placed on the automatic syringe, the needle is placed in the body as anatomically desired, and then the flow switch is opened and the fluid aspiration or biopsy is performed. Once done with the aspiration procedure, the flow switch is closed and the needle extracted; the closed flow switch prevents air and the tissue sample from being sucked into the syringe. To expel the sample, the flow switch is rotated and loosened to permit air to flow into the syringe barrel, and then is rotated the opposite direction to become airtight again. The locked plunger is rotated, releasing the plunger lock and permitting the coiled spring to act on the plunger and creating positive pressure in the syringe. The needle is then placed over a slide or specimen container, or in the case of aspirated fluid, into a vacuum tube, and the flow switch opened permitting active automatic expulsion of the sample. The procedure can then be repeated.

To automatically inject for nerve blocks, hydrodissection procedures, dilation of musculoskeletal structures, or to autodetect the epidural space, the syringe is first filled with fluid, typically saline or local anesthetic. First the syringe is prepared as above for an aspiration procedure. Next a needle is placed on the automatic syringe, and needle placed into the fluid that is to be injected, either a stoppered vial or other fluid reservoir. The flow switch is then opened and the syringe automatically fills with fluid. Once filled with fluid the flow switch is closed, the plunger is rotated to release the spring that then impels the plunger downward creating internal pressure within the syringe. The procedure needle is then placed on the automatic syringe and inserted in the body anatomic target. When the flow switch is opened, the syringe automatically injects while held in the finger grip position. Flow rate is controlled by needle length and diameter and by the operator, who can move the flow switch incrementally to different positions to control the flow. In the case of nerve blocks the automatic syringe injects local anesthetic under great control next to the target nerve, for hydrodissection the operator uses the automatic syringe to dissect anatomic planes and free trapped structures under image guidance, in the case of musculoskeletal injections, the target structure is dilated to create a space for corticosteroid or hyaluron, and after dilation, a syringe exchange is performed. In the case of autodetection, an epidural needle is placed into the interspinous ligament, the automatic syringe attached, the flow switch opened, and the needle advanced. When the syringe begins to automatically inject, the epidural space has been reached. This project complied with the Helsinki Declaration. Twenty-four knee arthrocenteses, 10 suction biopsies in various tissues, 10 hydrodissection procedures, and 5 nerve blocks (4 peripheral and 1 epidural) were performed by experienced proceduralists with sonographic guidance with automatic syringes in an IRB (internal review board)-approved protocol comparative trial registered at ClinicalTrials.gov (Clinical Trial Identifier NCT00651625). A portable ultrasound unit with a 10-5 MHz 38 mm broadband liner array transducer (Sonosite M-Turbo, SonoSite, Inc. 21919 30th Drive SE, Bothell, Wash. 98021, website: www.sonosite.com) was used.

Determination of Syringe and Needle Control: The validated quantitative needle-based linear displacement model method was used to measure ex vivo syringe needle control in both conventional syringes and automatic syringes. This method measures loss of control in the forward direction. This experimental variable has been validated to predict operator ability to control the needle, procedure time, procedure pain, needle trauma to patient tissues, hemorrhage, and procedure outcome in clinical syringe and needle procedures. A rigid polystyrene marker is placed on the needle to a preset indelible mark on the needle, and then the needle is advanced into a model target until the polystyrene marker is touching the surface of the target. The operator then performs the syringe procedure. Loss of control in the forward direction (unintended forward penetration) pushes the polystyrene marker posteriorly on the needle past the indelible mark, permitting precise measurement in mm of loss of control. The needle is marked every 10 mm, which allows precise measurement of loss of control in either the forward or reverse direction using metric calipers for <10 mm changes. Finally, the operator difficulty to perform each procedure was determined with the 10 cm visual analog scale for difficulty (VAS-D). Using the VAS-D, 0 cm=no difficulty and 10 cm=extreme difficulty.

Three different syringe procedures were performed using the displacement method in each the ml and 20 ml conventional and automatic syringes; 1) aspiration of 10 ml or 20 ml of fluid, respectively (typical of body fluid aspiration procedures), 2) injection of 10 ml or 20 ml of fluid, respectively (typical of nerve block, hydrodissection, musculoskeletal dilation, and body cavity autodetection procedures), and 3) needle control during a suction biopsy procedure. Each of these procedures was performed for total 10 times each with each syringe device. The order of each procedure with each syringe was randomized to minimize the possibilities of a consistent bias and bias associated with training effects. The conventional syringe was operated with 2 hands and the automatic syringe was operated with one hand while held in the pencil grip.

Statistical analysis: Data was entered into Excel and analyzed in SAS. Primary comparisons between the automatic syringe and the conventional syringe of the outcome variables, unintended penetration (mm), and operator difficulty (0-10 cm analog difficulty scale) were done using a 3-way ANOVA with type of syringe, procedure, and size of syringe as factors. Measurement data was compared post-hoc with the student t-test, and categorical data with Fisher's exact test. A power calculation was made using preliminary data at this level where α=0.0001, power=0.9, and allocation ratio=1.0 indicated that n=5 in each group would provide statistical power at the p<0.001 level and n=10 in each group at the p<0.0001 level.

Results: The automatic syringe could held in the pencil grip and controlled with one hand while operating the ultrasound probe with the other hand. The results show that as syringe size increased, forward penetration and difficulty with the conventional syringe consequently increased. However, the automatic syringes were able to reduced both forward penetration and difficulty even as syringe size increased (ANOVA, syringe-size interaction, p<0.0001).

During the aspiration of fluid procedures, compared to conventional syringes, the 10 ml and 20 ml automatic syringes reduced unintended penetration by 73%

(−3.1±0.6 mm) and 75% (−3.6±0.5 mm), respectively (each p<0.0001). When measuring operator difficulty using the 10 cm VAS-D, the 10 ml and 20 ml automatic syringes were easier to control compared to corresponding conventional syringes. The 10 ml and 20 ml automatic syringes reduced difficulty of aspiration of fluid compared to the corresponding conventional syringes by 77% (−4.7±0.6 cm) and −77% (−4.9±0.5 cm), respectively (p<0.0001).

When performing aspiration for biopsy procedures, automatic syringes again performed better in terms of control of the needle and syringe. Compared to conventional syringes, the 10 ml and 20 ml automatic syringes reduced unintended penetration by 71% (−2.9±0.6 mm) and 72% (−3.3±0.6 mm), respectively (each p<0.0001). When measuring operator difficulty using the 10 cm VAS-D, the 10 ml and 20 ml automatic syringes were much easier to control during aspiration biopsy compared to corresponding conventional syringes. The 10 ml and 20 ml automatic syringes reduced difficulty of the aspiration biopsy procedures compared to the corresponding conventional syringes by 69% (−2.7±0.4 cm) and 61% (−2.4±0.6 cm), respectively (p<0.0001).

Injection procedures were also better controlled with the automatic syringe. During injection procedures, compared to conventional syringes, the automatic syringes reduced unintended forward penetration in 10 ml and 20 ml syringes by 71% (−3.2±0.7 mm) and 67% (−2.9±0.6 mm), respectively (p<0.0001). The 10 ml and 20 ml automatic syringes reduced difficulty of the injection procedures compared to the corresponding conventional syringes by 71% (−3.2±0.5 cm) and 72% (−4.1±0.5 cm), respectively (p<0.0001). Thus, for aspiration of fluid, aspiration biopsy, and controlled injections, the automatic syringe enhanced needle control, one-handed operation, automatic injection and/or aspiration, and the ability to hold and control flow or vacuum while holding the syringe in the pencil grip.

Discussion: The present study demonstrates that finger-grip automatic syringes reduce dangerous unintended forward penetration of the needle, can be held with one hand in the pencil grip, are better controlled, and are less difficult to operate than conventional syringes for fluid aspiration, suction biopsy, and controlled injection procedures. The present study is the first report of the performance characteristics of a mechanical syringe that can both automatically aspirate and automatically inject, is operated with one hand, and is held in the finger tips in the pencil grip during sonographically guided procedures, including fluid aspiration and suction biopsy. This device can also administer controlled injections including administration of local anesthesia, nerve blocks, hydrodissection, musculoskeletal dilation, and autodetection of the epidural space. This type of device is another incremental development of adjunct syringe and needle technologies including syringe handles, syringe guns, and mechanical syringes intended to support and extend the effectiveness of image guidance, especially with the growth of dynamic image-guided aspiration and injection procedures where frequent needle positioning adjustment is required.

Ultrasound-directed needle and syringe procedures, originally pioneered by radiologists and obstetricians, are increasingly used throughout medicine, including critical care, emergency medicine, anesthesiology, nephrology, endocrinology, and the musculoskeletal clinic amongst others. Although radiologists traditionally have an ultrasound technician to assist them with the image-directed procedure, often other specialists who use sonography do not have the immediate assistance of an ultrasound technician, and thus often operate both the ultrasound probe and syringe themselves while frequently adjusting needle tip positioning while simultaneously aspirating or injecting, tasks that are difficult with one hand. Because of this, technologies that permit one-handed use of a syringe so that the free hand can operate the ultrasound probe are of interest. Such syringe adaptations include the addition of multiport valves, mechanical syringes, syringe holders, and vacuum syringes. These technologies are generally inexpensive, ranging from $0.50 US to $5.00 US per procedure representing 1% to 4% of the total costs of an image-guided procedure. Sollerman described the use of the dual syringe injector that permits a two-step injection procedure without changing syringes. Although useful to promote needle control during two-step injections of internal structures, this particular configuration does not permit aspiration. Other multiport valves have been used for similar purposes.

Another one-handed aspiration-injection syringe technology for ultrasound is the mechanical syringe that is operated with one hand, and injects by depressing one plunger and aspirates by depressing the other plunger, permitting sonographically guided procedures. Clinical trials have demonstrated that mechanical syringes are superior for aspiration-injection procedures to a conventional syringe, resulting in greater needle control, less procedural pain, greater aspirated fluid volume, less needle trauma, and a reduced incidence of hemorrhage while improving outcomes in a variety of procedures including thyroid aspiration, abscess drainage, fine needle aspiration biopsy, administration of local anesthesia, sclerotherapy, fat biopsy, central venous access and breast procedures. Mechanical syringes, although highly controlled one-handed devices, are manual devices and do not automatically inject or automatically aspirate, and are usually held in the syringe grip rather than the pencil grip.

Automatic aspiration is advantageous for suction biopsy and body fluid aspiration procedures because the syringe can be held passively in the fingers in the pencil-grip with enhanced control. One such aspirating technology is the vacuum syringe that is held in one hand in the pencil grip, and automatically aspirates for suction biopsy or body fluid aspiration, but cannot automatically inject because the plunger is fixed and no automatic drive mechanism is present. There are also other one-handed devices for needle procedures including syringe handles, syringe pistols, syringe pens, and syringe guns almost all of which automatically or manually aspirate, but generally do not have automatic injection capabilities. Because controlled injection is becoming increasingly important for image-guided procedures including nerve blocks, hydrodissection, dilation of musculoskeletal structures, and autodetection of body spaces, syringe technologies that can automatically inject have advantages. One-example is automatic injection pens that can be held with one hand, but these are one-way dose-delivery devices that do not permit precise control of injection flow and pressure, and generally do not have aspiration capabilities. Spring-loaded epidural space detection syringes can be held in the pencil grip and can automatically inject, but do not have automatic aspiration capabilities, are difficult to fill prior to use, do not lock the plunger, and have no mechanism to control of injection flow.

The present study demonstrates that an automatic syringe that is held in the pencil grip and is operated with one-hand, is better controlled and less difficult to operate than a conventional syringe for fluid aspiration, suction biopsy, and controlled injection procedures. The automatic syringe combines aspects of autoinjectors and epidural space detection syringes in the sense of a spring to drive the plunger, but unlike either of these technologies has a forward flow switch that can act as an on-off device, but also can be variably adjusted to precisely control fluid flow out of the syringe. Thus, when administering local or regional anesthesia, performing hydrodissection, dilating musculoskeletal structures prior to injection, and determining loss of resistance for identifying the epidural space, the switch permits precise on-off capabilities and real time adjustment of fluid flow. In addition, the automatic syringe has an on-off plunger lock, which permits stable vacuum within the barrel so that the syringe can automatically fill with saline or local anesthetic prior to use, and then by disengaging the plunger, permits release of the spring-generated force causing pressure in the syringe and fluid flow out of the barrel, which is then controlled by the forward flow switch. In contrast, autoinjectors are pre-filled and have no control of outflow. Unlike the automatic syringes, spring-loaded epidural space detection syringes possess neither forward on-off valve nor a mechanism to fix the plunger, thus, the operator must control the plunger to prevent premature injection of air or saline out of the syringe. Further, there is no mechanism for automatically filling the syringe, and, unlike the automatic syringe, the epidural detection syringes have no control of fluid outflow.

Automatic syringes also combine aspects of certain vacuum syringes including the ability to lock or otherwise restrain the plunger and to close the flow switch, permitting vacuum to reside in the syringe in a stable, secure fashion, and permitting precise release of vacuum for fluid aspiration or suction biopsy procedures. Vacuum syringes are used for amniocentesis, liposuction, fat biopsy, vacuum curettage, and fine-needle aspiration biopsy. Certain of these syringes can be held in stable pencil-grip positioning for aspiration, but cannot automatically inject, rather must be manually injected. Typically these syringes are either reusable and expensive in the range of $100 per syringe, have a syringe holder or gun that must be sterilized between uses, or are a modification of a conventional syringe that includes a plunger lock. The automatic syringe can function like a vacuum syringe, but unlike a vacuum syringe, the automatic syringe can automatically expel the sample of tissue or fluid after the procedure by rotating the plunger and releasing the spring, which then drives the plunger, creating pressure to expel the sample.

Specialty syringes have the disadvantages of restricted use to one or a few procedures, and thus, a low volume of manufacturing that increases costs, non-standard syringe parts, and a tendency to reuse rather than dispose of expensive devices. Further, for aspiration procedures, if the syringe volume is inadequate, multiple devices must be used further increasing procedural costs. For example, a 60 ml suction syringe for vacuum curettage costs $100 US per syringe, while a conventional 60 ml syringe costs $1 US per syringe. Thus, a low-cost disposable automatic syringe based on a conventional syringe that can be used for multiple different procedures may have economic advantages related to higher volume of production, and would cost in the range of $1 to $3/syringe—much less than contemporary vacuum syringes, electrically driven solenoid-based syringes, and loss of resistance syringes. Although automatic syringes slightly increase the costs of an image-guided procedure (about 1% to 3% of the cost of an image-guided procedure), the fact that mechanical syringes combined with ultrasound guidance have been shown to improve the outcome of image-guided procedures provides the promise that other highly controlled syringe devices like the automatic syringe may also enhance outcomes.

In summary, the present study demonstrates that automatic syringes that can be held the pencil grip provide one-handed operation and enhanced needle control during ultrasound-guided aspiration and injection procedures. The role of advanced syringe devices in sonographically-guided procedures will continue to be of interest as ultrasound technology further penetrates into day-to-day outpatient procedural medicine.

Large Volume Arthrocentesis. Ultrasound image-guidance results in less traumatic, more complete arthrocentesis. The present randomized controlled trial investigated new aspirating syringe technologies for image-guided arthrocentesis of the knee.

Materials and Methods: 42 ultrasound-guided arthrocentesis procedures of the knee were randomized to new aspirating technologies: 1) a 60 ml vacuum syringe, or 2) the 25 ml RPD (reciprocating procedure device) mechanical syringe. The one-needle two-syringe technique was used. Completeness of arthrocentesis was determined by sonography. Outcome measures included patient pain by the 10 cm Visual Analogue Pain Scale (VAS), synovial fluid volume, complications, and therapeutic outcome at 2 weeks. Needle control of the new technologies was measured in the linear displacement model and compared mechanical and vacuum syringes to conventional syringes.

Results: Both the mechanical syringe and the vacuum syringe controlled the needle better than a conventional syringe, reducing unintended forward penetration by 75% (3.6±0.5 mm) and 87% (12.0±4.2 mm), respectively (p<0.0001). Image-guided aspiration with both aspiration technologies permitted complete arthrocentesis with low levels of procedural pain (10 cm VAS; Vacuum Syringe: 2.9±3.1 cm, Mechanical Syringe: 3.1±2.5 cm, p>0.8) and significant fluid yield (Vacuum Syringe: VAS: 35±23 ml, Mechanical Syringe: 34±27 ml, p>0.9). The vacuum syringe permitted facile automatic aspiration of up to 60 mls; the mechanical syringe permitted 25 ml aspiration before a syringe exchange was required.

Conclusions: Ultrasound-guided arthrocentesis of the knee can be facilely achieved with new aspirating syringe technologies with improved needle control, enhanced patient safety, large volume aspiration, and complete joint decompression.

Arthrocentesis and its derivative intraarticular procedures are important outpatient invasive procedures in musculoskeletal medicine. Arthrocentesis is essential for the diagnosis of septic arthritis and inflammatory joint disease, and is the basic underlying procedure for intraarticular therapy, including therapeutic arthrocentesis, needle lavage, and intraarticular injection of therapeutic substances. Complete arthrocentesis before injection of corticosteroid or hyaluron confirms the diagnosis, eliminates the possibility of infection, reduces patient pain, and improves the response to the injected drug. Despite the importance of arthrocentesis to the diagnosis and management of conditions of the knee, arthrocentesis with conventional methods can be unsuccessful, painful, and unnecessarily traumatic.

Sonographic guidance is increasingly used in musculoskeletal medicine and has provided more accurate needle placement, and in some cases provides better outcomes and cost-effectiveness for both arthrocentesis and joint injections. Typically a 20 ml to 60 ml conventional syringe is used to aspirate large knee effusions, however, larger syringes require considerable hand strength and are difficult to control while operating the ultrasound probe with one hand and aspirating with a syringe with the other hand. One solution to this problem has been the development of one-handed mechanical syringes for aspiration and injection procedures. For a very large effusion, a vacuum bottle method has also been used; however, the setup for a vacuum bottle is awkward and unnecessarily large for the smaller 20 ml to 120 ml volumes typical of arthrocentesis of the knee. Thus, improved methods for one-operator ultrasound-guided arthrocentesis of the knee would be advantageous.

Vacuum syringes are used for suction biopsy, amniocentesis, suction curettage, and liposuction, but there are few if any reports of the use of vacuum syringes for arthrocentesis. We hypothesized that vacuum syringes and mechanical syringes that could be operated with one hand would be better controlled than conventional syringes during large-volume arthrocentesis, and that both new aspiration technologies would be effective for ultrasound-guided arthrocentesis of the knee. The present randomized, controlled trial compared the control characteristics of the conventional syringe, a mechanical syringe, and a vacuum syringe, and then determined the comparative effect of mechanical syringes and vacuum syringes on ultrasound-guided arthrocentesis.

Subjects: This project was in compliance with the Helsinki Declaration, approved by the institutional review board (IRB), and was registered at ClinicalTrials.gov (Clinical Trial Identifier NCT00651625). Inclusion criteria included: 1) palpable symptomatic effusion of the knee with suprapatellar distention, 2) indications for therapeutic-diagnostic arthrocentesis, 3) confirmation of significant synovial fluid in the suprapatellar bursa by sonographic interrogation, and 4) formal consent of the patient to undergo the procedure and participate in the research. A total of 42 knees with significant effusions with suprapatellar bursa distention were randomized between 1) a mechanical syringe (20 knees), or 2) a vacuum syringe (22 knees). 22 patients had rheumatoid arthritis and 20 osteoarthritis evenly distributed between the treatment groups.

Needle Introduction Technique for Arthrocentesis. The straight leg lateral suprapatellar bursa (superiolateral) approach was used to insert the needle and perform arthrocentesis under ultrasound image-guidance. Prior to the procedure, the presence of suprapatellar bursal distention was confirmed with sonographic interrogation using a portable ultrasound unit with a 10-5 MHz 38 mm broadband liner array transducer (Sonosite M-Turbo, SonoSite, Inc. 21919 30th Drive SE, Bothell, Wash. 98021, website: www.sonosite.com). The knee was placed in the extended position, and the ultrasound probe placed transversely over the quadriceps tendon to image the distended suprapatellar bursa.

The one-needle two-syringe technique was used where 1) one needle is used for anesthesia, arthrocentesis, and intraarticular injection; 2) a first syringe is used to anesthetize, aspirate effusion, and dilate the joint space, and 3) a second syringe is used to inject the intraarticular therapy. A 25 gauge 1.5 inch needle (305783-25 g1.5 inch BD Eclipse Safety Needle, BD, 1 Becton Drive, Franklin Lakes, N.J. 07417, website: http://www.bd.com) was mounted on a 5 ml mechanical syringe (RPD procedure syringe, AVANCA Medical Devices, Inc, Albuquerque, N. Mex., USA) filled with 5 ml of 1% lidocaine (Xylocalne® 1%, AstraZeneca Pharmaceuticals LP, 1800 Concord Pike, P.O. Box 15437, Wilmington, Del. 19850-5437). Using 25 gauge needle on the mechanical syringe, 3 ml of lidocaine was used to first anesthetize the skin, subcutaneous tissues and synovial membrane. The needle was then extracted and inactivated, and rotated off of the mechanical syringe. Subsequently a 1.5 inch 18 gauge needle was placed on the 5 ml mechanical syringe, and introduced into the knee using ultrasound image guidance and expelling the remaining 2 mls of lidocaine into the synovial membrane, and then the suprapatellar bursa was penetrated, the needle tip was visualized in the synovial pocket, and 5 ml of synovial fluid aspirated into the mechanical syringe. The 5 ml mechanical syringe was then rotated off of the intraarticular needle and the needle left in place.

Arthrocentesis with the Mechanical Syringe. The mechanical syringe for ultrasound-guided arthrocentesis was a 25 ml mechanical syringe (RPD 25 ml syringe, AVANCA Medical Devices, Inc, Albuquerque, N. Mex., USA. website: www.AVANCAMedical.com). The mechanical syringe is formed around the core of a conventional syringe barrel and plunger, but has a parallel accessory plunger and an accessory barrel to control the motion of the accessory plunger (FIG. 3). The two plungers are mechanically linked by a pulley in an opposing fashion, resulting in a set of reciprocating plungers. Thus, when the accessory plunger is depressed with thumb, the syringe aspirates, and when the dominant plunger is depressed with the thumb, the syringe injects. This permits the index and middle fingers to remain in one position during both aspiration and injection, while the thumb only needs to move in a horizontal plane to the alternative plunger in order to change the direction of aspiration or injection. Mechanical syringes permits greater control when used with sonography and easy detection of small amounts of synovial fluid that flash back into the barrel confirming true intraarticular positioning.

The 25 ml mechanical syringe was first cycled to break the bond of the plunger stopper with barrel, the air was then expelled. The mechanical syringe was then rotated on the indwelling needle (or the needle rotated on the syringe) and then the aspiration plunger was gently depressed. The mechanical syringe was then filled with 25 ml of synovial fluid. If the joint was not completed decompressed as visualized by ultrasound, the mechanical syringe was rotated off the intraarticular needle, and the syringe emptied into a sterile specimen container. The mechanical syringe was then reattached and filled again as above. After the joint was completely decompressed as possible by sonography, the mechanical syringe was rotated off of the needle, and a 3 ml conventional syringe prefilled with 80 mg triamcinolone acetonide suspension (Kenalog® 40, Westwood-Squibb Pharmaceuticals, Inc (Bristol-Myers Squibb), 345 Park Ave, New York, N.Y. 10154-0004, USA) was rotated onto the intraarticular needle, and the treatment was injected. The needle was then extracted, and firm pressure applied to the puncture site.

Arthrocentesis with the Vacuum Syringe. The vacuum syringe consisted of a conventional 60 ml syringe (BD 60 ml Syringe, Luer-Lok™ Tip, REF 309653, BD Franklin Lakes, N.J. 07417). This 60 ml conventional syringe was fitted with a sterile flow switch (QOSINA, Flow Control Switch, part number 97337, Quosina Corp., 150-Q Executive Drive, Edgewood, N.Y. 11717-8329 U.S.A), and a custom plunger lock (60 ml plunger lock). To create vacuum in the 60 ml conventional syringe, the flow switch was closed, the distal portion of the plunger lock fitted into the thumb rest of the plunger, the plunger pulled back to the 60 ml mark, and the proximal portion of the plunger lock fitted into the syringe barrel so that the plunger remained at the 60 ml plunger displacement mark. This 60 ml conventional vacuum syringe achieved a vacuum level of 650 Torr (mm Hg).

The 60 ml vacuum syringe was rotated onto the 18 gauge indwelling intraarticular needle. The flow switch was then opened and synovial fluid began flowing automatically into the syringe. Movement of fluid could be observed in the transparent portion of the flow switch. Then using ultrasound-guidance to position the needle tip away from the synovial membrane, villous excresences, and intrasynovial debris, the vacuum syringe was then filled automatically with up to 60 ml of fluid. If more fluid remained, the flow switch was closed, and the vacuum syringe and switch were rotated off of the indwelling needle. Then a second vacuum syringe was attached and the flow switch opened and the process was repeated. After complete arthrocentesis, the vacuum syringe was rotated off and corticosteroid injected as above.

Outcome Measures: Aspirated fluid volume was quantified in milliliters (ml). Patient pain was measured with the standardized and validated 0-10 cm Visual Analogue Pain Scale (VAS), where 0 cm=no pain and 10 cm=unbearable pain. Significant pain was defined as a VAS 5 cm. Pain by VAS was determined 1) prior to the procedure (baseline pain), 2) during arthrocentesis (procedural pain) and 3) 2 weeks post procedure (pain at primary outcome). Two weeks has been demonstrated as the outcome measurement time most likely to detect maximum clinical effect of injected corticosteroid; thus, the 2-week observation was considered the primary outcome measure.

Quantifying Syringe and Needle Control: he validated quantitative needle-based displacement model for syringe control was used to measure needle control in conventional, mechanical, and vacuum syringes. This model measures loss of control in either the forward or reverse direction. These variables have been validated to predict the outcomes of procedure time, patient pain, complications, needle trauma, hemorrhage, and physician satisfaction. The model consists of 1.3 cm thick open-cell flexible polystyrene foam, which is used to stimulate the target tissue. The foam layer is held together by Velcro restraints and is affixed to a rigid backing that is held upright with a rigid frame. A needle on the syringe was inserted into the foam layer. A rigid polystyrene marker is placed on the needle to a preset indelible mark on the needle, and then the needle is advanced into the foam until the polystyrene marker is touching the surface of the target tissue. The operator then performs the syringe procedure. Loss of control in the forward direction (unintended forward penetration) pushes the polystyrene marker posteriorly on the needle past the indelible mark, permitting precise measurement in mm of loss of control. The needle is marked every 10 mm, which allows precise measurement of loss of control in either the forward or reverse direction. For recording measurements less than 10 mm, a standard metric caliper was used. Finally, the operator recorded the difficulty of each procedure using the visual analog scale for difficulty (10 cm Difficulty-VAS) (14,25-27,40-42). Using the Difficulty-VAS, 0 cm=no difficulty and 10 cm=extreme difficulty.

Linear displacement model procedures: The linear displacement model measured syringe and needle control in a procedure typical of arthrocentesis, specifically aspiration of 25 mls of fluid with 25 ml mechanical syringe compared to 20 mls with the 20 ml conventional syringe, and aspiration of 60 mls of fluid the 60 ml vacuum syringe compared to the 60 ml conventional syringe. The procedures were performed for 10 cycles each with a conventional syringe operated with 2 hands and the mechanical and vacuum syringes operated using one hand. The order of each procedure with each syringe was randomized to reduce the possibility of a consistent bias or a bias from a training effect.

Statistical Analysis: Data were entered into Excel (Version 5, Microsoft, Seattle, Wash.), and analyzed in SAS (SAS/STAT Software, Release 6.11, Cary, N.C.). Differences between parametric two group data were determined with the t-test. Differences in categorical data were determined with Fisher's Exact Test, while differences between multiple parametric data sets were determined with Fishers Least Significant Difference Method. Correlations between parametric data were determined with logistic regression and between non-parametric data with Spearman correlation and Kendall rank method.

Results. The conventional syringe resulted in significant loss of needle control in the forward direction (unintended forward penetration) of 5 mm to 13 mm (60 ml conventional syringe: 13.4±2.3 mm; 20 ml conventional syringe: 4.8±0.6 mm). Both aspirating syringe technologies were better controlled and easier to operate with one-hand compared to the corresponding conventional syringe operated with 2 hands. The 25 ml RPD mechanical syringe reduced unintended forward penetration of the needle tip during aspiration by 75% (3.6±0.5 mm) (p<0.0001), and reduced the difficulty of aspiration by 64% (Change in Difficulty-VAS: 4.9±2.1 cm) (p<0.0001). The vacuum syringe was also superior to the conventional syringe, reducing unintended forward penetration of the needle tip during aspiration by 87% (12.0±4.2 mm), and reduced the difficulty of operating the syringe by 74% (Change in Difficulty-VAS: 7.2±1.6 cm) (p<0.0001). Thus, both aspirating syringe technologies, the vacuum syringe and mechanical syringe, were superior to the corresponding conventional syringe in needle control characteristics and ease of use.

Direct clinical comparisons between the mechanical syringe and vacuum syringe were made. Both technologies permitted facile large volume body fluid aspiration, and were equivalent in procedural pain, aspirated fluid volume, and pain outcome. However, a syringe exchange to fully drain the joint was necessary in only 18% of knee effusions aspirated with the 60 ml vacuum syringe, but was required in 50% of large knee effusions with the 25 ml mechanical syringe (p<0.035). In 2 cases the 18 gauge needle of the vacuum syringe became clogged with a loculated effusion, and could not be cleared without removing the syringe and/or plunger lock. In contrast, when the needle became clogged on the mechanical syringe, the needle could be easily cleared by depressing the injection plunger; then aspiration could be resumed by gently depressing the aspiration plunger.

Discussion. Ultrasound image-guidance has been demonstrated to be superior to palpation-guided methods for both arthrocentesis and intraarticular accuracy, and has recently been reported to significantly improve the outcomes and cost-effectiveness of intraarticular injections. However, for a single operator, large volume ultrasound-guided arthrocentesis of the knee can be challenging because the operator must operate the ultrasound probe with one hand and aspirate with a large volume syringe in the other. One-handed aspiration with a conventional syringe results in significant loss of needle control, and can result in increased procedural pain, increased needle damage to soft tissue and cartilage, and increased complications including hemorrhage.

The present study demonstrates that new aspirating syringe technologies, the mechanical syringe and the vacuum syringe, are better controlled, prevent dangerous unintended forward penetration, and are easier to operate than a conventional syringe for large volume fluid aspiration. Further, although previous studies have demonstrated that mechanical syringes are superior to conventional syringes for arthrocentesis and intraarticular injections, the present study demonstrates for the first time that ultrasound-guided arthrocentesis can be facilely achieved with one-handed with either a vacuum syringe or a mechanical syringe with generally equivalent outcomes including procedural pain, mean aspirated fluid volume, and therapeutic outcomes.

There were certain limitations to each device. A syringe exchange was necessary more often with the 25 ml mechanical syringe to fully decompress knee joints with >25 ml effusions. Additionally, the vacuum syringe could become clogged with loculated effusions in a minority of cases, and the needle was difficult to clear without removing the syringe while the mechanical syringe could be easily cleared by depressing the injection plunger. This study suggests that for ultrasound-guided arthrocentesis by a single operator, either of these new one-handed aspirating syringes provide greater needle control and patient safety, while facilely permitting effective arthrocentesis. For one-operator ultrasound-guided arthrocentesis of massive effusions without loculation, the 60 ml vacuum syringe is to be recommended; for smaller effusions or loculated effusions, the mechanical syringe is to be preferred; alternatively, the ultrasound probe can be operated by an assistant and a conventional syringe controlled by the primary operator, who can then use two hands. However, in most practices that use sonographic guidance in the clinic, one operator controls both the ultrasound probe and the aspirating syringe.

A number of technologies have been developed to assist in intraarticular injections and arthrocentesis, including multiport valves, mechanical syringes, sonographic guidance, and vacuum devices. Except for sonographic guidance that increases the cost of arthrocentesis by 260% ($183), these technologies are generally inexpensive, ranging from $0.30 US to $2.50 US representing 1% to 3% of the arthrocentesis procedure. Sollerman described the use of the dual syringe injector that permits a two-step injection procedure without changing syringes. Although very useful to promote needle control for the one-needle two-syringe technique and for hydrodissection prior to injection of bursa, tendon sheaths, and joints, this particular configuration does not permit aspiration, and thus is not useful for arthrocentesis as presently configured. Simkin and Gardner described the use of a 3-way stopcock for the one-needle two-syringe technique where a syringe exchange is not necessary. Although a stopcock is useful, the lack of control of the syringe during aspiration is transmitted through the stopcock to the needle; thus, a stopcock method would also benefit from better control of the syringe.

Draeger et al described the successful use of mechanical syringes for arthrocentesis, providing greater control, less trauma, and more complete joint decompression. Mechanical syringes have a reciprocating mechanism that prevents device lengthening during aspiration-injection, and permit better needle control and aspiration-injection characteristics. Clinical trials have demonstrated that mechanical syringes are superior for arthrocentesis to a conventional syringe, resulting in greater needle control, less procedural pain, greater aspirated fluid volume, less needle trauma, and a reduced incidence of hemorrhage. Clinical trials have also demonstrated that mechanical syringes improve the outcomes of intraarticular injection of corticosteroid or hyaluron. Mechanical syringes also provide greater safety and improved outcomes for other needle procedures, including thyroid aspiration procedures, suction biopsy, abscess aspiration, administration of local anesthesia, sclerotherapy, fat biopsy, central venous access, breast procedures, and ultrasound-guided procedures. The present study confirms that mechanical syringes are superior in control characteristics to conventional syringes, reducing the dangerous unintended forward penetration by 75% (3.6±0.5 mm) during an aspiration procedure, and further demonstrates that large volume arthrocentesis can be facilely performed with these devices, although syringe exchanges are required for volumes over 25 mls. Mechanical syringes also have the advantage that if clogging occurs, the needle can be facilely cleared during the procedure by depressing the injection plunger. Mechanical syringes contribute 2-3% ($1.50 to $2.00 US) to the 00084-50021; 40 cost of a palpation-guided arthrocentesis and 0.8% of a sonographically guided arthrocentesis, and because of improved outcomes and safety, have been demonstrated to be cost-effective.

Nahir et al described the use of vacuum devices for arthrocentesis, specifically a vacuum bottle for massive knee effusions. A vacuum bottle is effective for massive knee effusions, and an essentially identical setup has been used since 1954 for large volume thoracentesis, amniocentesis, and paracentesis. However, the 20 ml to 120 ml volumes typical of aspirable knee effusions are less than the 250 ml to 1000 ml volumes typical with vacuum bottle setups, thus, generally a vacuum bottle exceeds the demands of the procedure and the excessive vacuum often pulls the synovial membrane against the needle bevel, slowing or stopping aspiration. The vacuum bottle also isolates the fluid, making post-aspiration processing of the specimen more difficult, requiring the additional step of re-aspiration of fluid with a syringe.

Although not described specifically for arthrocentesis, vacuum syringes have been used for amniocentesis, liposuction, fat biopsy, vacuum curettage, and fine-needle aspiration biopsy. Typically these syringes are reusable and expensive in the range of $100 per syringe. The expense of these dedicated vacuum syringes are related to the low volume of production, the use of non-standard non-conventional syringes, the requirement that the devices be sturdy during the surgical procedure and not permit flexing, bending, or dislocation at the needle fitting, and the presence of non-standard non-luer fittings. All of these characteristics make these dedicated vacuum syringes unsuitable for arthrocentesis, which must utilize inexpensive technologies to be cost-effective.

In the present study, to keep costs low, a conventional 60 ml syringe out of the package (approximately $1.00 US per syringe) was used as the vacuum syringe. A flow valve (approximately $0.35 US per valve) and a custom plunger lock ($0.50 per plunger lock) were then added to the syringe. This type of simple vacuum syringe would thus increase the cost of arthrocentesis by a net of $0.75 US, approximately 1% of palpation-guided arthrocentesis and 0.3% of a sonographically guided arthrocentesis, yet the vacuum syringe permits one-handed operation and automatic aspiration of up to 60 ml of fluid. Alternative designs of plunger locks that require special integrated parts, that are internalized into the syringe barrel, require changes in the design of the plunger, or require assembly before the syringe is packaged all could have functional advantages, but have a tendency to increase costs due to low-volume production, the need for new precise molds, additional assembly costs, and the inability to use low-cost off-the-shelf sterile syringes. The type of vacuum syringe with an integrated plunger lock would make sense if the same vacuum syringe technology were used frequently in other aspiration procedures (amniocentesis, thoracentesis, paracentesis, fat biopsy, fine needle aspiration biopsy, etc) so that the volume of production would lower costs; for arthrocentesis alone, as shown here, the use of a off-the-shelf sterile conventional 60 ml syringe with simple application of an external valve and plunger lock is probably the most cost-effective approach.

The present study demonstrates that vacuum syringes are superior in control characteristics to conventional syringes during aspiration reducing the dangerous unintended forward penetration by 87% (12.0±4.2 mm) during an aspiration procedure, and further demonstrates that large volume arthrocentesis up to 60 ml can be facilely performed automatically with these devices without a syringe exchange. The study also demonstrates for the first time that the 60 ml vacuum syringe provides outcomes equivalent to that obtained with a highly controlled mechanical syringe, a syringe technology that has been proven to improve outcomes of intraarticular procedures relative to conventional syringes. Although useful in greater than 90% of large effusions, the vacuum syringe technology failed in loculated effusions where needle obstruction with synovial fluid debris occurred, and, unlike mechanical syringes, were difficult to clear without removing the syringe or plunger lock, which can be hazardous during a procedure. However, loculated effusions can be easily detected prior to the procedure by ultrasound, and appear as echoreflective punctate, particulate, or fibrinous material suspended in synovial fluid. Thus, vacuum syringes should not be used for known loculated effusions; rather for loculated effusions a one-handed mechanical syringe should be considered for a single-operator ultrasound-guided procedure or a conventional syringe for a two-operator ultrasound-guided procedure.

In summary, ultrasound-guided arthrocentesis of the knee can be facilitated with new aspirating syringe technologies that provide improved needle control, enhanced patient safety, one-handed operation, and facile large-volume aspiration.

The present invention has been described as set forth herein in relation to various example embodiments and design considerations. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1) A syringe that can aspirate and can inject, comprising:
a) a barrel having a needle fitting mounted with a first end and finger flanges mounted with a second end;
b) a plunger subsystem comprising a plunger body having a first cross-sectional area suitable for disposition within the barrel; a plunger flange providing a thumb rest mounted with a first end of the plunger body; a stopper-seal complex mounted with a second end of the plunger body and configured to slidingly engage and seal with the barrel when the plunger body is disposed within the barrel and having a cross-sectional area;
c) a mechanical locking mechanism that reversibly engages the plunger subsystem and the barrel such that motion of the plunger subsystem relative to the barrel is inhibited when the locking mechanism is engaged and allowed with the locking mechanism is disengaged.

2) A syringe as in claim 1, wherein the plunger subsystem is prevented from moving toward the first end of the barrel when the locking mechanism is engaged.

3) A syringe as in claim 1, wherein the needle fitting comprises a leur fitting.

4) A syringe as in claim 1, wherein plunger body defines one more notches or recesses therein, and wherein the locking mechanism comprises an endplate mounted with the second end of the barrel and configured such that the endplate engages one or more of the notches or recesses when the plunger body is at a first rotational orientation relative to the barrel, and such that the endplate does not engage a notch or recess when the plunger body is at a second rotational orientation relative to the barrel.

5) A syringe as in claim 4, wherein the plunger body has a first cross-sectional dimension intersecting a notch or recess, and a second cross-sectional dimension not intersecting a notch or recess, wherein the first cross-sectional dimension is less than the second-cross-sectional dimension, and wherein the first cross-sectional dimension plus the depth of the notch or recess is greater than the second cross-sectional dimension.

6) A syringe as in claim 1, further comprising a finger-operable valve mounted with the needle fitting such that passage of material into or out of the barrel is controlled by the valve.

7) A syringe as in claim 7, wherein the finger-operable valve is chosen from the group consisting of a slide valve, a modified stopcock, a turn cock, a return valve, a pinch valve, and a pin valve.

8) A syringe as in claim 1, wherein the locking mechanism comprises a substantially rigid element configured to mount with the finger flanges of the barrel and with the plunger subsystem when the plunger body is disposed partly within the barrel, such that motion of the plunger subsystem into the barrel is inhibited by the locking mechanism when mounted with the finger flanges and with the plunger subsystem.

9) A syringe as in claim 8, wherein the substantially rigid element has a forward surface, fitting, or notch that abuts the opening or finger flanges of the barrel, and a rearward surface, fitting, or notch that receives, abuts and engages the thumbrest of the plunger subsystem.

10) A syringe as in claim 9, wherein the engagement of the substantially rigid member of the plunger subsystem includes a force-bearing surface of sufficient size to prevent damage to the plunger subsystem from forces transmitted vie the substantially rigid member.

11) A syringe as in claim 8, wherein the substantially rigid element has a forward surface, fitting, or notch that abuts the opening or finger flanges of the barrel, a surface, fitting, or notch that receives, abuts and engages the thumbrest of the plunger and a slit than engages a strut or vane of the plunger body to prevent relative rotation of the plunger subsystem and the locking mechanism.

12) A syringe as in claim 8, wherein the substantially rigid element has a forward surface, fitting, or notch that abuts the opening or finger flanges of the barrel, a surface, fitting, or notch that receives, abuts and engages the thumbrest of the plunger and a hand or finger grip on the locking mechanism to assist in pulling back the plunger and inserting the plunger lock.

13) A syringe as in claim 8, wherein the substantially rigid element comprises one or more resilient tines that can be forced inward to reside next to the plunger within the barrel and that can be reversibly compressed and released so that the forward surface, fitting, or notch of the tine abuts the opening or finger flanges of the barrel, and distal a surface, fitting, or circular structure that receives, abuts and engages the thumbrest of the plunger.

14) A syringe as in claim 1, further comprising a spring mounted with the plunger subsystem and with the barrel when the plunger body is disposed within the barrel such that the spring exerts force between the plunger subsystem and the second end of the barrel.

15) A syringe as in claim 14, wherein the plunger subsystem is prevented from moving toward the first end of the barrel when the locking mechanism is engaged.

16) A syringe as in claim 14, wherein the needle fitting comprises a leur fitting.

17) A syringe as in claim 14, wherein plunger body defines one more notches or recesses therein, and wherein the locking mechanism comprises an endplate mounted with the second end of the barrel and configured such that the endplate engages one or more of the notches or recesses when the plunger body is at a first rotational orientation relative to the barrel, and such that the endplate does not engage a notch or recess when the plunger body is at a second rotational orientation relative to the barrel.

18) A syringe as in claim 17, wherein the plunger body has a first cross-sectional dimension intersecting a notch or recess, and a second cross-sectional dimension not intersecting a notch or recess, wherein the first cross-sectional dimension is less than the second-cross-sectional dimension, and wherein the first cross-sectional dimension plus the depth of the notch or recess is greater than the second cross-sectional dimension.

19) A syringe as in claim 14, further comprising a finger-operable valve mounted with the needle fitting such that passage of material into or out of the barrel is controlled by the valve.

20) A syringe as in claim 19, wherein the finger-operable valve is chosen from the group consisting of a slide valve, a modified stopcock, a turn cock, a return valve, a pinch valve, and a pin valve.

21) A syringe as in claim 14, wherein the locking mechanism comprises a substantially rigid element configured to mount with the finger flanges of the barrel and with the plunger subsystem when the plunger body is disposed partly within the barrel, such that motion of the plunger subsystem into the barrel is inhibited by the locking mechanism when mounted with the finger flanges and with the plunger subsystem.

22) A syringe as in claim 21, wherein the substantially rigid element has a forward surface, fitting, or notch that abuts the opening or finger flanges of the barrel, and a rearward surface, fitting, or notch that receives, abuts and engages the thumbrest of the plunger subsystem.

23) A syringe as in claim 22, wherein the engagement of the substantially rigid member of the plunger subsystem includes a force-bearing surface of sufficient size to prevent damage to the plunger subsystem from forces transmitted vie the substantially rigid member.

24) A syringe as in claim 21, wherein the substantially rigid element has a forward surface, fitting, or notch that abuts the opening or finger flanges of the barrel, a surface, fitting, or notch that receives, abuts and engages the thumbrest of the plunger and a slit than engages a strut or vane of the plunger body to prevent relative rotation of the plunger subsystem and the locking mechanism.

25) A syringe as in claim 21, wherein the substantially rigid element has a forward surface, fitting, or notch that abuts the opening or finger flanges of the barrel, a surface, fitting, or notch that receives, abuts and engages the thumbrest of the plunger and a hand or finger grip on the locking mechanism to assist in pulling back the plunger and inserting the plunger lock.

26) A syringe as in claim 21, wherein the substantially rigid element comprises one or more resilient tines that can be forced inward to reside next to the plunger within the barrel and that can be reversibly compressed and released so that the forward surface, fitting, or notch of the tine abuts the opening or finger flanges of the barrel, and distal a surface, fitting, or circular structure that receives, abuts and engages the thumbrest of the plunger.

27) A syringe as in claim 14, wherein the plunger subsystem is urged toward the first end of the barrel by the spring.

28) A method of assembling a syringe as in claim 14, comprising mounting the spring with the plunger body, inserting the plunger body and spring into the barrel, compressing the spring, and engaging the locking mechanism.
